(19) United States
(12) Patent Application Publication
(10) Pub. No.: US 2025/0364078 A1
(43) Pub. Date: Nov. 27, 2025

(54) METHOD FOR PERFORMING LOCAL ALIGNMENT, METHOD OF VARIANT CALLING, AND PROCESSING DEVICE AND SYSTEM FOR FACILITATING VARIANT CALLING

(71) Applicant: National Yang Ming Chiao Tung University, Hsinchu City (TW)

(72) Inventors: Bo-Cheng LAI, Hsinchu City (TW); Aman SINHA, Xinfeng Township (TW)

(73) Assignee: National Yang Ming Chiao Tung University, Hsinchu City (TW)

(21) Appl. No.: 18/763,773

(22) Filed: Jul. 3, 2024

(30) Foreign Application Priority Data
May 27, 2024 (TW) 113119481

(51) Int. Cl. G16B 30/10; G16B 20/00

(52) U.S. Cl. CPC G16B 30/10; G16B 20/00

(57) ABSTRACT

A method for performing local alignment based on a query sequence of DNA and a reference sequence of DNA includes: obtaining a bit matrix H; determining at least one diagonal based on the bit matrix H; for each of the at least one diagonal, calculating an initial score for the diagonal, determining at least one trace region, determining a sub-alignment for each of the at least one trace region, consolidating the diagonal and the sub-alignment respectively of the at least one trace region to obtain an alignment, and obtaining an alignment score based on the initial score and the partial score respectively of the sub-alignment respectively of the at least one trace region; and among each of the at least one alignment thus determined respectively for each of the at least one diagonal, reserving one of the at least one alignment that has the highest alignment score therefrom.

### CROSS-REFERENCE TO RELATED APPLICATION

This application claims priority to Taiwanese Invention Patent Application No. 113119481, filed on May 27, 2024, and incorporated by reference herein in its entirety.

### FIELD

The disclosure relates to a method for performing local alignment, a method of variant calling, a processing device for facilitating variant calling, and a system for facilitating variant calling.

### BACKGROUND

Next-generation sequencing (NGS) has been used to determine the order of nucleotides in entire genomes or target regions of deoxyribonucleic acid (DNA), and variant calling aims to identify variants, e.g., single nucleotide polymorphisms (SNPs) and small insertions and deletions (indels), from DNA sequencing data obtained by using NGS. The GATK HaplotypeCaller is a genomic analysis toolkit for variant calling based on an approach of de-novo assembly, which includes steps of: identifying an active region; determining haplotypes by assembly of the active region; determining likelihoods of the haplotypes; and assigning a genotype to a sample based on the likelihoods. Conventionally, the step of determining haplotypes is implemented by merely using a Smith-Waterman algorithm.

### SUMMARY

Therefore, an object of the disclosure is to provide a method for performing local alignment, a method of variant calling, a processing device for facilitating variant calling, and a system for facilitating variant calling.

According to a first aspect of the disclosure, the method for performing local alignment is implemented based on a query sequence of deoxyribonucleic acid (DNA) and a reference sequence of DNA. The method includes steps of:
obtaining a bit matrix H that has an X number of rows each of which is related to a nucleotide of the query sequence of DNA, and a Y number of columns each of which is related to a nucleotide of the reference sequence of DNA, a cell H(m,n) of the bit matrix H having a value of one when an m^{th} one of nucleotides of the query sequence of DNA is identical to an n^{th} one of nucleotides of the reference sequence of DNA, and having a value of zero when the m^{th} one of nucleotides of the query sequence of DNA is different from the n^{th} one of nucleotides of the reference sequence of DNA, where X and Y are each a positive integer, m is an integer ranging from zero to (X−1), and n is an integer ranging from zero to (Y−1);
determining, based on the bit matrix H, at least one diagonal that contains d number of consecutive cells from H(m,n) to H(m+d−1,n+d−1) of the bit matrix H, where d is a positive integer greater than a preset length threshold and represents a length of said at least one diagonal;
for each of said at least one diagonal that contains a start cell H(m,n) and an end cell H(m+d−1,n+d−1),
   calculating an initial score for the diagonal according to a predetermined scoring scheme of sequence alignment,
   determining at least one trace region in the bit matrix H, said at least one trace region being defined by trace bounds that include one of a first pair of cells of the bit matrix H and a second pair of cells of the bit matrix H, the first pair of cells including the start cell H(m,n) of the diagonal and a start cell H(0,0) of the bit matrix H, the second pair of cells including the end cell H(m+d−1,n+d−1) of the diagonal and an end cell H(X−1,Y−1) of the bit matrix H,
   for each of said at least one trace region, determining a sub-alignment that is one of paths passing through cells in the trace region from an upper-left corner to a lower-right corner of the trace region, and that has a highest partial score among all of the paths according to the predetermined scoring scheme of sequence alignment,
   consolidating the diagonal and the sub-alignment respectively of said at least one trace region to obtain an alignment, and
   obtaining an alignment score based on the initial score and the partial score respectively of the sub-alignment respectively of said at least one trace region; and
among each of the at least one alignment thus determined respectively for each of said at least one diagonal, reserving one of the at least one alignment that has the highest alignment score therefrom, said one of the at least one alignment thus reserved being related to a haplotype.

According to a second aspect of the disclosure, the method of variant calling includes steps of:
identifying an active region on a reference sequence of DNA according to a plurality of overlapping reads that are obtained in DNA sequencing, each of the overlapping reads representing a sequence that overlaps with the active region of the reference sequence of DNA;
generating a de Brujin graph (DBG) based on the overlapping reads and the active region of the reference sequence of DNA;
performing sequential traversal on the DBG to determine a query sequence of DNA;
obtaining a bit matrix H that has an X number of rows each of which is related to a nucleotide of the query sequence of DNA, and a Y number of columns each of which is related to a nucleotide of a reference sequence of DNA, a cell H(m,n) of the bit matrix H having a value of one when an m^{th} one of nucleotides of the query sequence of DNA is identical to an n^{th} one of nucleotides of the reference sequence of DNA, and having a value of zero when the m^{th} one of nucleotides of the query sequence of DNA is different from the n^{th} one of nucleotides of the reference sequence of DNA, where X and Y are each a positive integer, m is an integer ranging from zero to (X−1), and n is an integer ranging from zero to (Y−1);
determining, based on the bit matrix H, at least one diagonal that contains d number of consecutive cells from H(m,n) to H(m+d−1,n+d−1) of the bit matrix H, where d is a positive integer greater than a preset length threshold and represents a length of said at least one diagonal;
for each of said at least one diagonal that contains a start cell H(m,n) and an end cell H(m+d−1,n+d−1),
   calculating an initial score for the diagonal according to a predetermined scoring scheme of sequence alignment,
   determining at least one trace region in the bit matrix H, said at least one trace region being defined by trace bounds that include one of a first pair of cells of the bit matrix H and a second pair of cells of the bit matrix H, the first pair of cells including the start cell H(m,n) of the diagonal and a start cell H(0,0) of the bit matrix H, the second pair of cells including the end cell H(m+d−1,n+d−1) of the diagonal and an end cell H(X−1,Y−1) of the bit matrix H,
   for each of said at least one trace region, determining a sub-alignment that is one of paths passing through cells in the trace region from an upper-left corner to a lower-right corner of the trace region, and that has a highest partial score among all of the paths according to the predetermined scoring scheme of sequence alignment,
   consolidating the diagonal and the sub-alignment respectively of said at least one trace region to obtain an alignment, and
   obtaining an alignment score based on the initial score and the partial score respectively of the sub-alignment respectively of said at least one trace region;
among each of the at least one alignment thus determined respectively for each of said at least one diagonal, reserving one of the at least one alignment that has the highest alignment score therefrom, said one of the at least one alignment thus reserved being related to a haplotype;
determining read-haplotype likelihoods by using a pair hidden Markov model (pair-HMM) forward algorithm (PFA) based on the haplotype and the overlapping reads; and
determining a genotype by using Bayes' Theorem based on the read-haplotype likelihoods thus determined.

According to a third aspect of the disclosure, the processing device is adapted to be electrically connected to a central processing unit (CPU). The processing device includes a programmable core, a vector coprocessor electrically connected to the programmable core, and an L1-cache electrically connected to the programmable core and the vector coprocessor. The L1-cache is configured to store input data offloaded by the CPU. The input data contains information about an active region of a reference sequence of DNA and a plurality of overlapping reads that are obtained in DNA sequencing. Each of the overlapping reads represents a sequence that overlaps with the active region of the reference sequence of DNA. The programmable core is configured to read the input data from the L1-cache, and to send the input data to the vector coprocessor. The vector coprocessor is configured to perform k-mer hashing and unique k-mer detection based on the input data, and to store results respectively of k-mer hashing and unique k-mer detection in the L1-cache, where k is a positive integer. The programmable core is configured to read the results of k-mer hashing and unique k-mer detection from the L1-cache, to generate a DBG based on the results of k-mer hashing and unique k-mer detection, to perform sequential traversal on the DBG to determine a query sequence of DNA, and to store information about the query sequence of DNA in the L1-cache.

According to a fourth aspect of the disclosure, the system includes a CPU, and a distributed processing module electrically connected to the CPU. The distributed processing module includes a plurality of first processing devices and a plurality of second processing devices. The CPU is configured to identify an active region on a reference sequence of DNA according to a plurality of overlapping reads that are obtained in DNA sequencing, and to offload input data to the first processing devices of the distributed processing module. Each of the overlapping reads represents a sequence that overlaps with the active region of the reference sequence of DNA. The input data contains information about the active region of the reference sequence of DNA and the overlapping reads. The first processing devices of the distributed processing module are configured to generate a DBG based on the overlapping reads and the active region of the reference sequence of DNA, and to perform sequential traversal on the DBG to determine a query sequence of DNA. The CPU is further configured to obtain information about the query sequence of DNA from the first processing devices of the distributed processing module, and to send the input data and the information about the query sequence of DNA to the second processing devices of the distributed processing module. The second processing devices of the distributed processing module are configured to perform local alignment to determine a haplotype based on the query sequence of DNA and the reference sequence of DNA, and to determine read-haplotype likelihoods by using a PFA based on the haplotype and the overlapping reads. The CPU is further configured to determine a genotype by using Bayes' Theorem based on the read-haplotype likelihoods thus determined.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a flow chart illustrating a method of variant calling according to an embodiment of the disclosure.

FIG. 2 is a flow chart illustrating a procedure of constructing a bin-index table according to an embodiment of the disclosure.

FIG. 3 is a block diagram illustrating a system for facilitating variant calling according to an embodiment of the disclosure.

FIG. 4 is a block diagram illustrating a bit-matrix aligner of a second processing device of the system according to an embodiment of the disclosure.

FIG. 5 is a circuit diagram illustrating a processing engine of the bit-matrix aligner according to an embodiment of the disclosure.

FIG. 6 is a schematic diagram illustrating clusters of the second processing devices according to an embodiment of the disclosure.

FIGS. 7 to 9 are schematic diagrams cooperatively illustrating an example of k-mer hashing according to an embodiment of the disclosure.

FIG. 10 is a schematic diagram illustrating a row-major inter-task PFA scheme according to an embodiment of the disclosure.

FIGS. 11 to 16 are schematic diagrams cooperatively illustrating an example of performing local alignment by using a bit matrix according to an embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

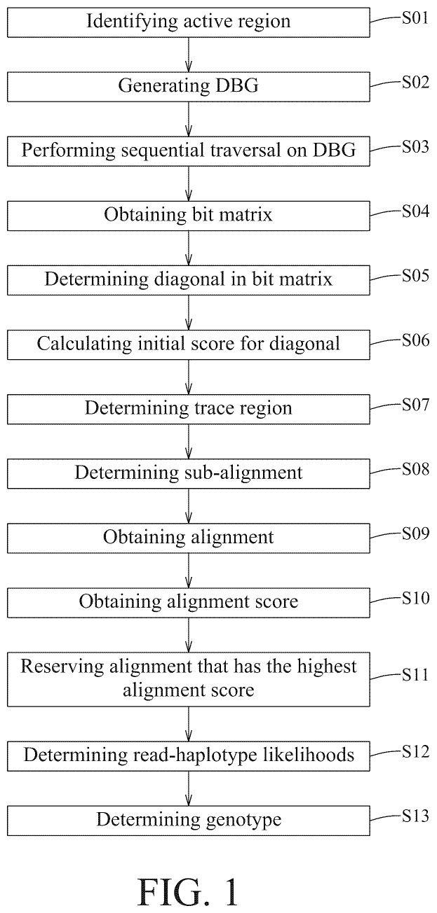

Referring to FIG. 1, an embodiment of a method of variant calling according to the disclosure is illustrated. The method includes steps S01 to S13 delineated below.

In step S01, an active region on a reference sequence of deoxyribonucleic acid (DNA) is identified according to a plurality of overlapping reads that are obtained in DNA sequencing. Each of the overlapping reads represents a sequence that overlaps with the active region of the reference sequence of DNA.

In step S02, a de Brujin graph (DBG) is generated based on the overlapping reads and the active region of the reference sequence of DNA.

In step S03, sequential traversal is performed on the DBG to determine a query sequence of DNA.

Since techniques related to identifying the active region, generating the DBG and performing sequential traversal on the DBG have been well known to one skilled in the relevant art, detailed explanation of the same is omitted herein for the sake of brevity.

Subsequently, steps S04 to S11 are executed for performing local alignment based on the query sequence of DNA and the reference sequence of DNA.

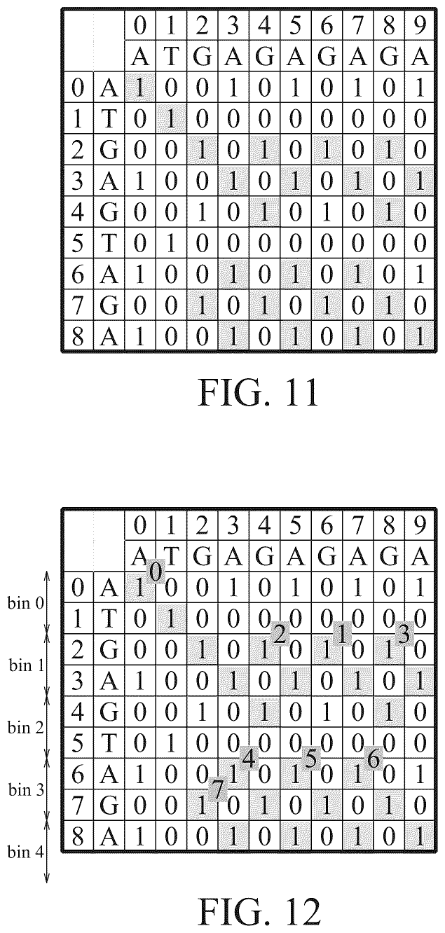

In step S04, a bit matrix H is obtained. The bit matrix H has an X number of rows each of which is related to a nucleotide of the query sequence of DNA, and a Y number of columns each of which is related to a nucleotide of a reference sequence of DNA, where X and Y are each a positive integer. A cell H(m,n) of the bit matrix H has a value of one when an m^{th} one of nucleotides of the query sequence of DNA is identical to an n^{th} one of nucleotides of the reference sequence of DNA, and has a value of zero when the m^{th} one of nucleotides of the query sequence of DNA is different from the n^{th} one of nucleotides of the reference sequence of DNA, where m is an integer ranging from zero to (X−1), and n is an integer ranging from zero to (Y−1). An example of the bit matrix H is illustrated in FIG. 11, where X is equal to nine and Y is equal to ten.

In step S05, based on the bit matrix H, at least one diagonal that contains d number of consecutive cells from H(m,n) to H(m+d−1,n+d−1) of the bit matrix H is determined, where d is a positive integer greater than a preset length threshold (e.g., two) and represents a length of said at least one diagonal.

For each of said at least one diagonal that contains a start cell H(m,n) and an end cell H(m+d−1,n+d−1), steps S06 to S10 are executed.

In step S06, an initial score is calculated for the diagonal according to a predetermined scoring scheme of sequence alignment. In this embodiment, the predetermined scoring scheme of sequence alignment is identical to a scoring scheme used in the GATK HaplotypeCaller (which is a conventional genomic analysis toolkit for variant calling), and has settings of scoring parameters as follows: a match condition corresponds to a score of 200; a mismatch condition corresponds to a score of −150; a gap-open condition corresponds to a score of −260; and a gap-extension condition corresponds to a score of −11. Since the predetermined scoring scheme of sequence alignment has been well known to one skilled in the relevant art, detailed explanation of the same is omitted herein for the sake of brevity.

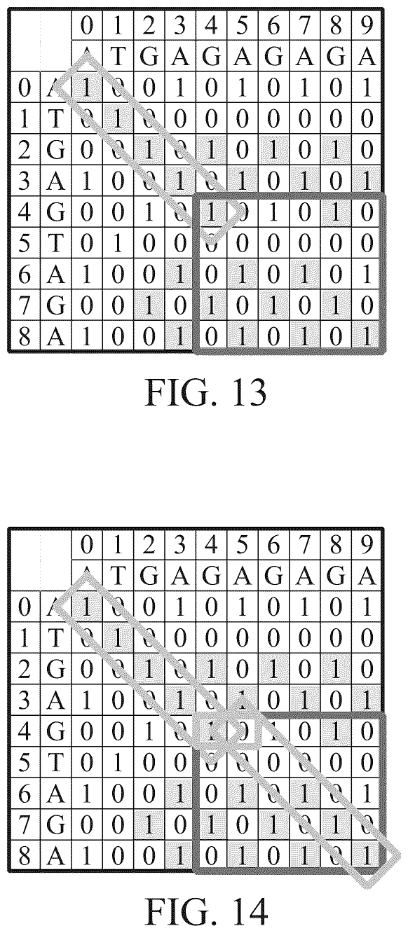

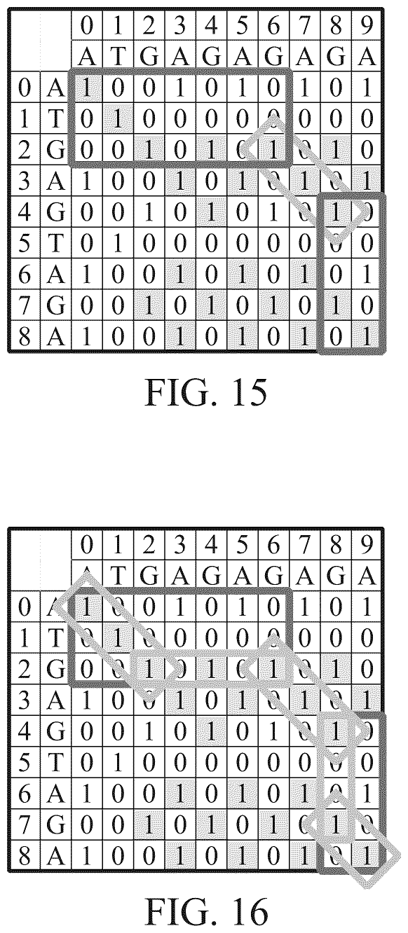

In step S07, at least one trace region is determined in the bit matrix H. Said at least one trace region is a rectangular region defined by trace bounds that include one of a first pair of cells of the bit matrix H and a second pair of cells of the bit matrix H. The first pair of cells include the start cell H(m,n) of the diagonal and a start cell H(0,0) of the bit matrix H, and the second pair of cells include the end cell H(m+d−1,n+d−1) of the diagonal and an end cell H(X−1,Y−1) of the bit matrix H. In an example illustrated in FIG. 13, one trace region (which is surrounded by a dark-colored rectangle) is determined for one of the diagonals (which is surrounded by a light-colored rectangle). In another example illustrated in FIG. 15, two trace regions (which are respectively surrounded by two dark-colored rectangles) are determined for another of the diagonals (which is surrounded by a light-colored rectangle).

In step S08, for each of said at least one trace region, a sub-alignment is determined. The sub-alignment is one of paths passing through cells in the trace region from an upper-left corner (i.e., the start cell H(0,0) of the bit matrix H of the first pair of cells or the end cell H(m+d−1,n+d−1) of the diagonal of the second pair of cells) to a lower-right corner (i.e., the start cell H(m,n) of the diagonal of the first pair of cells or the end cell H(X−1,Y−1) of the bit matrix H of the second pair of cells) of the trace region, and has a highest partial score among all of the paths according to the predetermined scoring scheme of sequence alignment. In one embodiment, the sub-alignment is determined based on brute-force search or exhaustive search. Since techniques related to determining the sub-alignment has been well known to one skilled in the relevant art, detailed explanation of the same is omitted herein for the sake of brevity. Referring to FIGS. 14 and 16, sub-alignments that are surrounded with light-colored rectangles are determined in the trace regions.

In step S09, the diagonal and the sub-alignment respectively of said at least one trace region are consolidated to obtain an alignment. The alignment corresponds to a path passing through cells of the bit matrix H from the start cell H(0,0) of the bit matrix H to the end cell H(X−1,Y−1) of the bit matrix H.

In step S10, an alignment score is obtained based on the initial score and the partial score respectively of the sub-alignment respectively of said at least one trace region.

In step S11, among each of the at least one alignment thus determined respectively for each of said at least one diagonal, one of the at least one alignment that has the highest alignment score is reserved therefrom. Said one of the at least one alignment thus reserved is related to a haplotype.

In step S12, read-haplotype likelihoods are determined by using a pair hidden Markov model (pair-HMM) forward algorithm (PFA) based on the haplotype and the overlapping reads.

In step S13, a genotype is determined by using Bayes' Theorem based on the read-haplotype likelihoods thus determined.

Since techniques related to determining the read-haplotype likelihoods by using the PFA and determining the genotype by using Bayes' Theorem have been well known to one skilled in the relevant art, detailed explanation of the same is omitted herein for the sake of brevity.

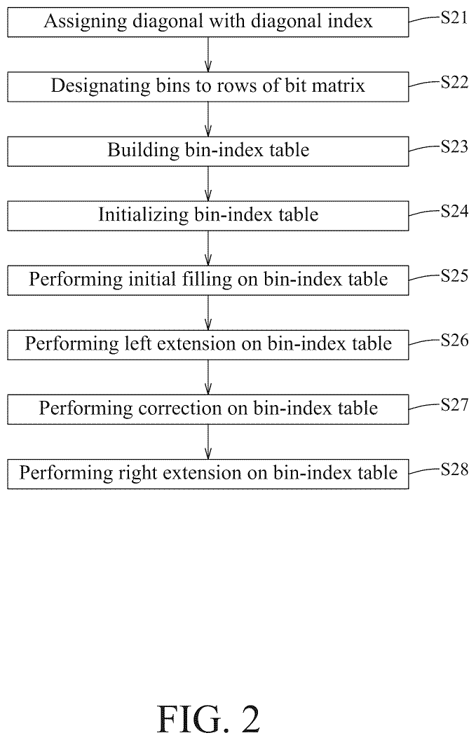

In order to enhance efficiency of performing the method that is previously described, a variant embodiment of the method of variant calling incorporates a data structure of a bin-index table that helps search for said at least one diagonal which has been determined in step S05 of the previous method. Since the variant embodiment is similar to the previous embodiment, descriptions regarding steps of the variant embodiment that are identical to those of the previous embodiment are not repeated, and only differences between the variant embodiment and the previous embodiment are explained in the following paragraphs for the sake of brevity. The variant embodiment of the method further includes, prior to step S06, steps S21 to S28 as shown in FIG. 2 and delineated below.

In step S21, for each of said at least one diagonal, the diagonal is assigned with a diagonal index that is an integer according to an ascending order of a row of the start cell of the diagonal, an ascending order of the length of the diagonal, and an ascending order of a column of the end cell of the diagonal. Referring to an example illustrated in FIG. 12, eight diagonals therein are respectively assigned with eight diagonal indexes from zero to seven (the diagonals are marked with symbols “0”, “1”, “2”, “3”, “4”, “5”, “6” and “7” respectively therebeside for illustration). Data related to the eight diagonals are summarized in Table 1 below.

TABLE 1

| | | | | | | | | |
|---|---|---|---|---|---|---|---|---|
| Diagonal index | 0 | 1 | 2 | 3 | 4 | 5 | 6 | 7 |
| Row of start cell | 0 | 2 | 2 | 2 | 6 | 6 | 6 | 7 |
| Row of end cell | 4 | 4 | 3 | 3 | 8 | 8 | 8 | 8 |
| Column of end cell | 4 | 8 | 5 | 9 | 5 | 7 | 9 | 3 |
| Length | 5 | 3 | 2 | 2 | 3 | 3 | 3 | 2 |

In step S22, a series of bins are designated to the X number of rows of the bit matrix H in a manner that every consecutive b number of rows from the start of the rows of the bit matrix H are designated as a respective one of the bins and remaining one(s) of the rows of the bit matrix H are designated as a last one of the bins, where b is a positive integer (e.g., two) and represents a bin size. Referring to FIG. 12, five bins (from bin 0 to bin 4) are designated to eight rows of the bit matrix H.

In step S23, the bin-index table is built. The bin-index table records a start index and an end index for each of the bins.

In step S24, the bin-index table is initialized by assigning, for each of the bins, a positive predetermined value to the start index and a negative predetermined value to the end index. In this embodiment, the positive predetermined value is exemplarily positive infinity and the negative predetermined value is exemplarily negative infinity. However, in other embodiments, the positive predetermined value may be an extreme large positive value and the negative predetermined value may be an extreme large negative value. An example of the bin-index table that has been initialized is shown in Table 2 below.

TABLE 2

| Bin | 0 | 1 | 2 | 3 | 4 |
|---|---|---|---|---|---|
| Start index | ∞ | ∞ | ∞ | ∞ | ∞ |
| End index | −∞ | −∞ | −∞ | −∞ | −∞ |

In step S25, initial filling is performed on the bin-index table. Specifically, for each of the bins in the bin-index table, the start index of the bin is updated with the diagonal index of a first one of said at least one diagonal having the end row that is designated to the bin, and the end index of the bin is updated with the diagonal index of a last one of said at least one diagonal having the start row that is designated to the bin. A result of performing initial filling on the bin-index table in Table 2 according to the example illustrated in FIG. 12 is shown in Table 3 below.

TABLE 3

| Bin | 0 | 1 | 2 | 3 | 4 |
|---|---|---|---|---|---|
| Start index | ∞ | 2 | 0 | ∞ | 4 |
| End index | 0 | 3 | −∞ | 7 | −∞ |

In step S26, left extension is performed on the bin-index table. Specifically, for each of the bins having the start index that is equal to the positive predetermined value, the start index of the bin is updated with the start index of a closest subsequent one of the bins having the start index that is not equal to the positive predetermined value. A result of performing left extension on the bin-index table in Table 3 is shown in Table 4 below.

TABLE 4

| Bin | 0 | 1 | 2 | 3 | 4 |
|---|---|---|---|---|---|
| Start index | 2 | 2 | 0 | 4 | 4 |
| End index | 0 | 3 | −∞ | 7 | −∞ |

In step S27, correction is performed on the bin-index table. Specifically, for each of the bins having the start index that is greater than the start index of a subsequent one of the bins, the start index of the bin is updated with the start index of the subsequent one of the bins. A result of performing correction on the bin-index table in Table 4 is shown in Table 5 below.

TABLE 5

| Bin | 0 | 1 | 2 | 3 | 4 |
|---|---|---|---|---|---|
| Start index | 0 | 0 | 0 | 4 | 4 |
| End index | 0 | 3 | −∞ | 7 | −∞ |

In step S28, right extension is performed on the bin-index table. Specifically, for each of the bins having the end index that is equal to the negative predetermined value, the end index of the bin is updated with the end index of a closest prior one of the bins having the end index that is not equal to the negative predetermined value. A result of performing right extension on the bin-index table in Table 5 is shown in Table 6 below.

TABLE 6

| Bin | 0 | 1 | 2 | 3 | 4 |
|---|---|---|---|---|---|
| Start index | 0 | 0 | 0 | 4 | 4 |
| End index | 0 | 3 | 3 | 7 | 7 |

In step S08 of the variant embodiment, a determination as to whether the trace region covers one of said at least one diagonal is made. In response to the trace region covering one of said at least one diagonal, the sub-alignment is created such that the sub-alignment includes at least a portion of said one of said at least one diagonal which is located within the trace region. In response to the trace region not covering any one of said at least one diagonal, the sub-alignment is created by using a Smith-Waterman algorithm. Since the Smith-Waterman algorithm has been well known to one skilled in the relevant art, detailed explanation of the same is omitted herein for the sake of brevity.

It is worth to note that for a trace region defined by a first cell H(m₁, n₁) and a second cell H(m₂, n₂) (i.e., the trace bounds), a range of the diagonal indexes to be searched for is from the start index of bin S to the end index of bin T, where S is an integer equal to a quotient of a row element m₁ of the first cell H(m₁, n₁) divided by the bin size, and T is an integer equal to a quotient of a row element m₂ of the second cell H(m₂, n₂) divided by the bin size. Referring to FIG. 12, several examples of utilizing the bin-index table shown in Table 6 to search for the diagonals shown in Table 1 are provided in the following for explanation. For a trace region defined by cells H(0, 0) and H(8, 9), a range of the diagonal indexes (shown in Table 1) to be searched for is from the start index of bin 0 (because the quotient of the row element of the cell H(0, 0), i.e., 0, divided by the bin size, i.e., 2, is equal to zero, i.e., 0/2=0) to the end index of bin 4 (because the quotient of the row element of the cell H(8, 9), i.e., 8, divided by the bin size of two is equal to four, i.e., 8/2=4). Similarly, for a trace region defined by cells H(4, 4) and H(8, 9), a range of the diagonal indexes to be searched for is from the start index of bin 2 (because the quotient of the row element of the cell H(4, 4), i.e., 4, divided by the bin size of two is equal to two, i.e., 4/2=2) to the end index of bin 4 (because the quotient of the row element of the cell H(8, 9), i.e., 8, divided by the bin size of two is equal to four, i.e., 8/2=4). For a trace region defined by cells H(4, 4) and H(6, 5), a range of the diagonal indexes to be searched for is from the start index of bin 2 (because the quotient of the row element of the cell H(4, 4), i.e., 4, divided by the bin size of two is equal to two, i.e., 4/2=2) to the end index of bin 3 (because the quotient of the row element of the cell H(6, 5), i.e., 6, divided by the bin size of two is equal to three, i.e., 6/2=3). For a trace region defined by cells H(0, 0) and H(2, 6), a range of the diagonal indexes to be searched for is from the start index of bin 0 (because the quotient of the row element of the cell H(0, 0), i.e., 0, divided by the bin size of two is equal to zero, i.e., 0/2=0) to the end index of bin 1 (because the quotient of the row element of the cell H(2, 6), i.e., 2, divided by the bin size of two is equal to one, i.e., 2/2=1).

An example of determining two alignments respectively for two diagonals in the bit matrix H according to FIG. 12 is shown in Table 7 below. Expressions “\hat{ }5M*$”, “\hat{ }*3M*S”, “\hat{ }*3M*S”, “1M1I3M”, “2M4D1M”, “3I2M”, “1M1I3M” “\hat{ }2M4D2M3I2M$” are regular expressions to specify alignment conditions, wherein “M” stands for the match condition, “D” stands for the deletion condition, and “I” stands for the insertion condition.

TABLE 7

| | | |
|---|---|---|
| Diagonal index | 0 | 1 |

| | | | |
|---|---|---|---|
| Trace direction | Right | Left | Right |
| Initial alignment | \hat{ }5M*$ | \hat{ }*3M*$ | \hat{ }*3M*$ |
| condition | | | |
| Initial score | 1000 | 600 | 600 |
| Trace bounds | H(4, 4); | H(0, 0); | H(4, 8); |
| | H(8, 9) | H(2, 6) | H(8, 9) |
| Traced alignment | 1M1I3M | 2M4D1M | 3I2M |
| condition | | | |
| Partial score | 540 | 307 | 118 |

| | | |
|---|---|---|
| Alignment score | 1340 | 625 |
| Consolidated | \hat{ }5M1I3M$ | \hat{ }2M4D2M3I2M$ |

alignment
condition

An example of consolidating the diagonal and the sub-alignments to obtain the alignment and consolidating the initial score and the partial scores to obtain the alignment score is provided herein for explanation. Referring to Table 7, the diagonal having the diagonal index of one corresponds to an initial alignment condition of “\hat{ }*3M*$” and an initial score of 600, a left sub-alignment corresponds to a trace alignment condition of “2M4D1M” and a partial score of 307, and a right sub-alignment corresponds to a trace alignment condition of “312M” and a partial score of 118. For left-side consolidation where the diagonal and the left sub-alignment are to be consolidated to result in a temporary alignment, the initial alignment condition “\hat{ }*3M*$” can be expressed as “NNMMMNNNN”, and the trace alignment condition “2M4D1M” of the left sub-alignment can be expressed as “MMDDDDM”, wherein “N” stands for a not-yet aligned nucleotide of the query sequence of DNA, and a bolded font symbol (i.e., “M”) stands for an overlapping nucleotide. A temporary consolidated alignment condition corresponding to the temporary alignment is expressed as “MMDDDDMMMNNNN”, and a temporary consolidated alignment score corresponding to the temporary alignment is calculated as (307−200)+600=707, where the subtraction of 200 is attributed to a superfluous count of the match condition (which corresponds to the score of 200). For right-side consolidation where the temporary alignment and the right sub-alignment are to be consolidated to result in a final consolidated alignment, the temporary consolidated alignment condition is expressed as “MMDDDDMMMNNNN”, and the trace alignment condition “312M” of the right sub-alignment can be expressed as “IIIMM”. A final consolidated alignment condition corresponding to the final consolidated alignment is expressed as “MMDDDDMMIIIMM” (i.e., “\hat{ }2M4D2M3I2M$” as shown in Table 7), and a final consolidated alignment score corresponding to the final consolidated alignment is calculated as (707−200)+118=625, where the subtraction of 200 is attributed to a superfluous count of the match condition.

Unlike the GATK haplotypecaller which merely uses the Smith-Waterman algorithm to perform the local alignment for determining a haplotype, the method according to the disclosure realizes the local alignment further based on the bit matrix H, which enables in-cache processing and minimizes memory-access overheads, reducing memory consumption for hardware implementing variant calling. In addition, operations performed based on the bit matrix H increases DRAM row-buffer hits, and thus improves memory-access patterns. Moreover, compared to sequential traceback in the Smith-Waterman algorithm, faster tracebacks may be achieved in the method according to the disclosure, so for performance of variant calling, latency may be reduced and throughput may be upgraded. Further, hardware resource requirements for implementing the method according to the disclosure is low because there is no need for multiple large adders, which are usually required by conventional Smith-Waterman hardware accelerators.

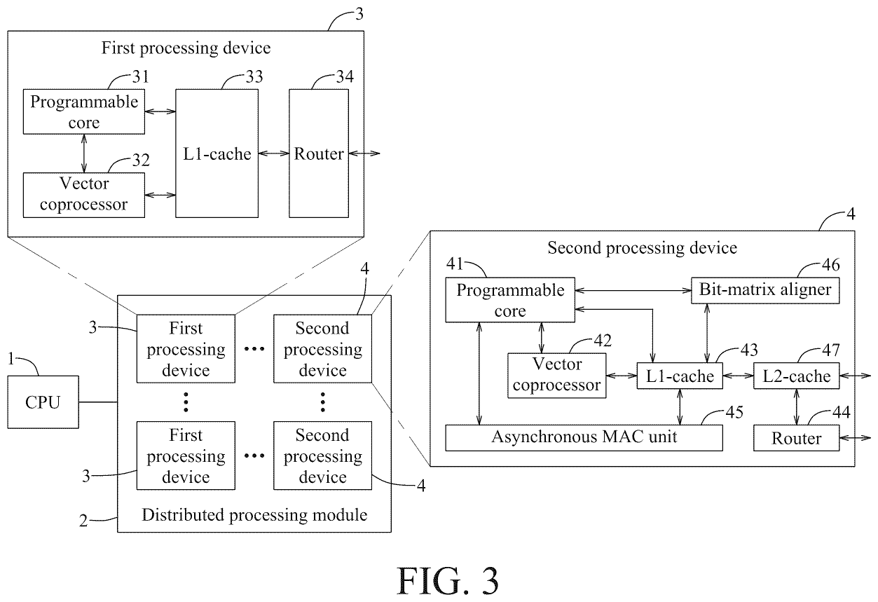

Referring to FIG. 3, an embodiment of a system for facilitating variant calling is illustrated. The system includes a central processing unit (CPU) 1, and a distributed processing module 2 that is electrically connected to the CPU 1. The CPU 1 may be implemented by a multithreaded CPU, but is not limited thereto. The distributed processing module 2 includes a plurality of first processing devices 3 and a plurality of second processing devices 4.

Each of the first processing devices 3 of the distributed processing module 2 is adapted to be electrically connected to the CPU 1, and includes a router 34 for packet switching. Similarly, each of the second processing devices 4 of the distributed processing module 2 is adapted to be electrically connected to the CPU 1, and includes a router 44 for packet switching. It is worth to note that each of the first processing devices 3 and the second processing devices 4 is assigned with a unique identification code, and the CPU 1 is configured to record the unique identification code(s) respectively of desired one(s) of the first processing devices 3 and the second processing devices 4, and to communicate with the desired one(s) of the first processing devices 3 and the second processing devices 4 based on the unique identification code(s) thus recorded. Since technologies about using packet switching for communication in an integrated circuit have been well known to one skilled in the relevant art, detailed explanation of the same is omitted herein for the sake of brevity.

The CPU 1 is configured to identify the active region on the reference sequence of DNA according to the overlapping reads that are obtained in DNA sequencing, and to offload input data to the first processing devices 3 of the distributed processing module 2. The input data contains information about the active region of the reference sequence of DNA and the overlapping reads.

The first processing devices 3 of the distributed processing module 2 are configured to generate the DBG based on the overlapping reads and the active region of the reference sequence of DNA, and to perform the sequential traversal on the DBG to determine the query sequence of DNA.

The CPU 1 is further configured to obtain information about the query sequence of DNA from the first processing devices 3 of the distributed processing module 2, and to send the input data and the information about the query sequence of DNA to the second processing devices 4 of the distributed processing module 2.

The second processing devices 4 of the distributed processing module 2 are configured to perform the local alignment to determine the haplotype based on the query sequence of DNA and the reference sequence of DNA, and to determine the read-haplotype likelihoods by using the PFA based on the haplotype and the overlapping reads.

The CPU 1 is further configured to determine the genotype by using Bayes' Theorem based on the read-haplotype likelihoods thus determined.

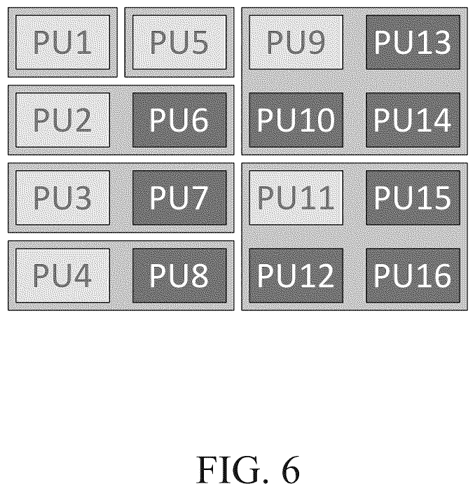

In one embodiment, the CPU 1 is configured to dynamically divide the second processing devices 4 into clusters of various sizes according to sizes respectively of the query sequence of DNA and the reference sequence of DNA (which can be used to estimate latencies for accomplishing tasks). It should be noted that each of the second processing devices 4 belongs only to one of the clusters. Further, the CPU 1 is configured to designate, for each of the clusters, one second processing device 4 in the cluster as a cluster manager, and to distribute tasks to the cluster managers respectively of the clusters. Each of the distributed tasks has a scale that positively correlates with a cluster size of the cluster of the cluster manager that was distributed with the task. For example, in this embodiment, the scale may be an estimated latency for accomplishing the task. Then, the cluster manager in each of the clusters would further distribute the task to other second processing devices 4 in the same cluster. It should be noted that since metadata related to tasks are transferred only among the cluster managers respectively of the clusters rather than among all of the second processing devices 4, network-on-chip (NoC) traffic overhead may be reduced. For example, as illustrated in FIG. 6, the distributed processing module 2 includes sixteen of the second processing devices 4 (which are respectively represented by sixteen blocks respectively denoted with “PU0” to “PU16”). Two of the sixteen second processing devices 4 (i.e., the blocks denoted with “PU1” and “PU5”) belong respectively to two small clusters, i.e., each of the small clusters includes only one second processing devices 4. Six of the sixteen second processing devices 4 (i.e., the blocks denoted with “PU2”, “PU3”, “PU4”, “PU6”, “PU7” and “PU8”) belong to three medium clusters, and each of the medium clusters includes two second processing devices 4. Eight of the sixteen second processing devices 4 (i.e., the blocks denoted with “PU9” to “PU16”) belong to two large clusters, and each of the large clusters includes four second processing devices 4. The CPU 1 is configured to designate one second processing device 4 in each of the small clusters, the medium clusters and the large clusters as the cluster manager, and to distribute a task to the cluster manager of one of the small clusters, the medium clusters and the large clusters according to a scale of the task. The white blocks denoted with “PU1” and “PU5” represent the cluster managers respectively of the small clusters. The white blocks denoted with “PU2”, “PU3” and “PU4” represent the cluster managers respectively of the medium clusters. The white blocks denoted with “PU9” and “PU11” represent the cluster managers respectively of the large clusters. It is worth to note that the way of dividing the second processing devices 4 into clusters (also known as cluster configuration) is based on historical observation data, an example of which is shown in Table 8 below, and the cluster configuration is not limited to the disclosure herein and may vary based on practical requirements. In one embodiment, the CPU 1 is configured to assign a task to one of the clusters that has a smallest size among available/soon-available and competent ones of the clusters based on an estimated latency of the task. That is to say, a smallest available/soon-available cluster would be assigned for any task that has a low estimated latency or a high sequential execution pattern. Because of features of load-balancing and scalability of the second processing devices 4 of the distributed processing module 2, minor NoC traffic overheads would incur, and full utilization of the second processing devices 4 may be ensured.

An estimated latency for a task of diagonal computation in local alignment or for a task of PFA may be determined based on a sequence size, and an estimated latency for a task of tracebacks in local alignment may be determined based on a diagonal coverage fraction in the bit matrix H (hereinafter also referred to as a diagonal coverage). An example of task distribution is shown in Table 9 below. Tasks “Aln-DP-T1” and “Aln-DP-T2” respectively represent two tasks of diagonal computation in local alignment, wherein estimated latencies are determined based on sequence sizes. Tasks “PFA-DP-T3” and “PFA-DP-T4” respectively represent two tasks of PFA, wherein estimated latencies are determined based on sequence sizes. Tasks “Aln-BT-T1” and “Aln-BT-T2” respectively represent two tasks of tacebacks in local alignment, wherein estimated latencies are determined based on diagonal coverages.

TABLE 8

| | | | | | | | |
|---|---|---|---|---|---|---|---|
| Maximum size of | 32 | 64 | 128 | 256 | 512 | 1024 | 2048 |
| sequence | | | | | | | |
| Occurrence proportion | 0.10 | 0.15 | 0.25 | 0.25 | 0.10 | 0.10 | 0.05 |
| of sequence | | | | | | | |
| Weightage | 0.01 | 0.03 | 0.09 | 0.18 | 0.14 | 0.28 | 0.28 |
| Cluster manager | PU1 | PU5 | PU4 | PU2 | PU3 | PU9 | PU11 |

TABLE 9

| | | Sequence | Cluster | Estimated | Diagonal |
|---|---|---|---|---|---|
| Time | Task | size | manager | latency | coverage |
| 0 | Aln-DP-T1 | 36 | PU1 | 162 | NA |
| 4 | Aln-DP-T2 | 52 | PU5 | 338 | NA |
| 11 | PFA-DP- | 18 | PU4 | 608 | NA |
| | T3 | | | | |
| 87 | PFA-DP- | 20 | PU1 | 72 | NA |
| | T4 | | | | |
| 163 | Aln-BT-T1 | 36 | PU2 | 807 | 0.8 |
| 339 | Aln-BT-T2 | 52 | PU5 | 9943 | 0.4 |

As shown in FIG. 3, the first processing device 3 further includes a programmable core 31, a vector coprocessor 32 electrically connected to the programmable core 31, and an L1-cache 33 electrically connected to the programmable core 31, the vector coprocessor 32 and the router 34.

The L1-cache 33 is configured to store the input data offloaded by the CPU 1. The programmable core 31 is configured to read the input data from the L1-cache 31, and to send the input data to the vector coprocessor 32. The vector coprocessor 32 is configured to perform k-mer hashing and unique k-mer detection based on the input data, and to store results respectively of k-mer hashing and unique k-mer detection in the L1-cache 33, where k is a positive integer (e.g., four). The programmable core 31 is configured to read the results of k-mer hashing and unique k-mer detection from the L1-cache 33, to generate the DBG based on the results of k-mer hashing and unique k-mer detection, to perform sequential traversal on the DBG to determine the query sequence of DNA, and to store information about the query sequence of DNA in the L1-cache 33. Since k-mer hashing and unique k-mer detection have been well known to one skilled in the relevant art, detailed explanation of the same is omitted herein for the sake of brevity. An example for brief explanation is provided as follows.

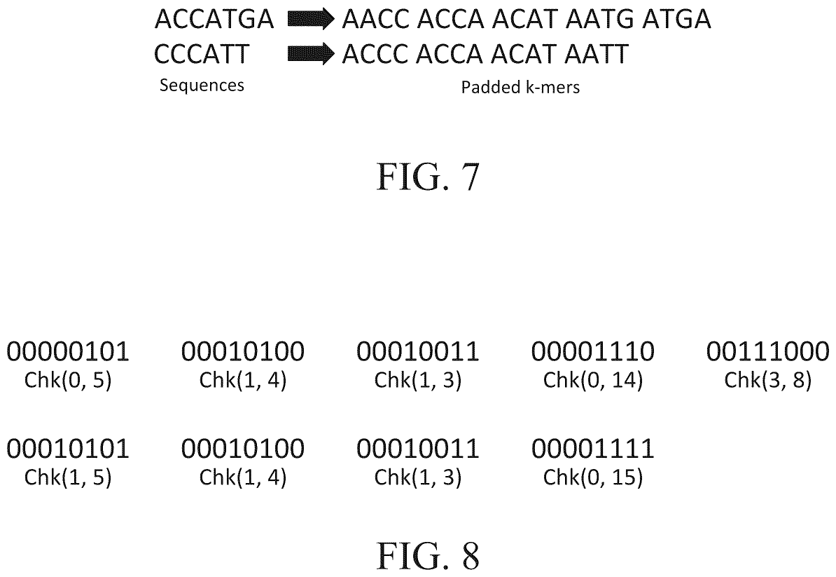

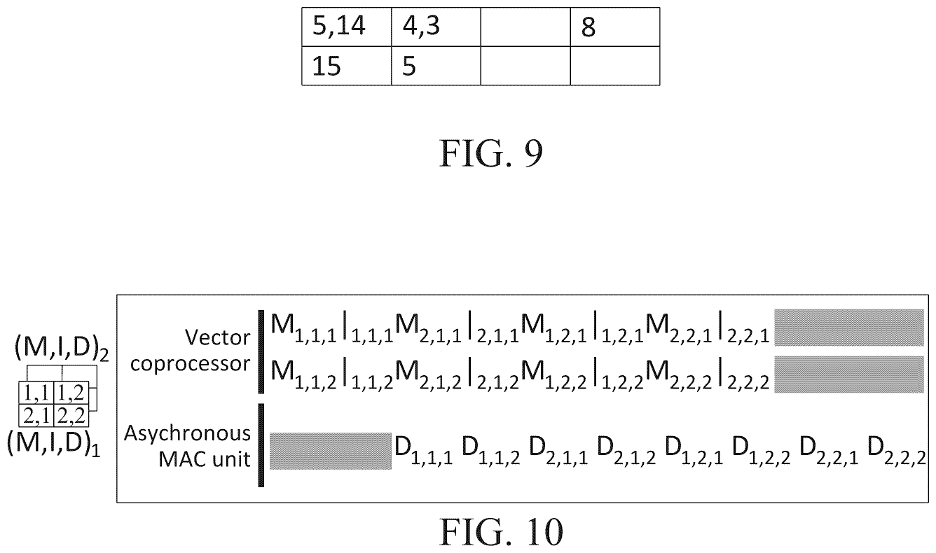

Referring to FIG. 7, for each of the overlapping reads, the vector coprocessor 32 is configured to select a plurality of sub-sequences from a sequence represented by the overlapping read using a sliding window of size three such that each of the sub-sequences contains three of consecutive nucleotides in the sequence. For each of sub-sequences, the vector coprocessor 32 is configured to pad the sub-sequence with an additional predetermined nucleotide (e.g., “A” as shown in FIG. 7) before the three of the consecutive nucleotides to generate a 4-mer. Referring to FIG. 8, the vector coprocessor 32 is further configured to encode each of the 4-mers that are generated respectively for the sub-sequences into an eight-bit binary sequence, wherein the eight-bit binary sequence represents a hash value (i.e., “Chk(x,y)” as shown in FIG. 8, where “x” and “y” are each an integer) that includes a pair of numbers (i.e., “x” and “y” in “Chk(x,y)”). A set of first four bits of the eight-bit binary sequence represents one of the pair of numbers of the hash value (i.e., “x” in “Chk(x,y)”), and a set of last four bits of the eight-bit binary sequence represents another of the pair of numbers of the hash value (i.e., “y” in “Chk(x,y)”). It is worth to note that in this embodiment, a nucleotide “A” of a 4-mer is encoded as a binary sequence “00”; a nucleotide “C” of a 4-mer is encoded as a binary sequence “01”; a nucleotide “G” of a 4-mer is encoded as a binary sequence “10”; and a nucleotide “T” of a 4-mer is encoded as a binary sequence “11”. For example, for the eight-bit binary sequence “00010100”, the first four bits “0001” thereof represent “1” of the hash value “Chk(1,4)”, and the last four bits “0100” thereof represent “1” of the hash value “Chk(1,4)”. For another example, for the eight-bit binary sequence “00010011”, the first four bits “0001” thereof represent “1” of the hash value “Chk(1,3)”, and the last four bits “0011” thereof represent “3” of the hash value “Chk(1,3)”. Then, the vector coprocessor 32 is configured to build a hash table that has a plurality of rows each having a plurality of sectors. Referring to FIG. 9, the hash table exemplarily includes two rows, and each of the rows has four sectors each having an identical size (i.e., having the same space for storage). It is worth to note that the one of the pair of numbers of the hash value (i.e., “x” in “Chk(x,y)”) is related to a sector number of one of the sectors of the hash table, and said another of the pair of numbers of the hash value (i.e., “y” in “Chk(x,y)”) is related to a value to be recorded in the hash table. For each of the eight-bit binary sequences corresponding respectively to the 4-mers, the vector coprocessor 32 is further configured to assign said another of the pair of numbers of the hash value (e.g., “4” of the hash value “Chk(1,4)” or “3” of the hash value “Chk(1,3)”) represented by the eight-bit binary sequence to one of the sectors of the hash table, and to record k-mer data related to the 4-mer that corresponds to the eight-bit binary sequence in the one of the sectors of the hash table. The k-mer data contains information about the 4-mer and a nucleotide to which the three number of the consecutive nucleotides corresponding to the 4-mer are to transit. It should be noted that in case that a sector within one of the rows of the hash table gests full, such case will be recorded and a next one of the rows would be allocated for recording the k-mer data. Furthermore, the vector coprocessor 32 is configured to search for the k-mer data recorded in the hash table by using 64-bit comparisons. It is worth to note that the scheme disclosed herein allows for k-mers of length up to 32.

Referring back to FIG. 3, the second processing device 4 further includes a programmable core 41, a vector coprocessor 42 electrically connected to the programmable core 41, an L1-cache 43 electrically connected to the programmable core 41 and the vector coprocessor 42, an asynchronous medium access control (MAC) unit 45 electrically connected to the programmable core 41 and the L1-cache 43, a bit-matrix aligner 46 electrically connected to the programmable core 41 and the L1-cache 43, and an L2-cache 47 electrically connected to the L1-cache 43 and the router 44. It is worth to note that the L2-cache 47 is shared among the second processing devices 4 and is configured to support operations involved in the local alignment and the PFA.

The L1-cache 43 is configured to store the input data offloaded by the CPU 1.

The programmable core 41 is configured to generate bit vectors based on the reference sequence of DNA contained in the input data, and to send the bit vectors to the bit-matrix aligner 46.

The bit-matrix aligner 46 is configured to generate the bit matrix H on-the-fly based on the bit vectors and the query sequence of DNA, to determine at least one diagonal of the bit matrix H, and to store said at least one diagonal in the L1-cache 43. It is worth to note that in this implementation, the bit matrix H is an intermediate representation which does not exist physically and does not have to be stored, thereby saving hardware resources for storing the bit matrix H.

For each of said at least one diagonal, the programmable core 41 is configured to calculate the initial score for the diagonal according to the predetermined scoring scheme of sequence alignment, and to determine said at least one trace region in the bit matrix H. For each of said at least one trace region, the programmable core 41 is configured to determine the sub-alignment. Then, the programmable core 41 is configured to consolidate the diagonal and the sub-alignment respectively of said at least one trace region to obtain the alignment, and to obtain the alignment score based on the initial score and the partial score respectively of the sub-alignment respectively of said at least one trace region.

Among each of the at least one alignment thus determined respectively for each of said at least one diagonal, the programmable core 41 is configured to reserve one of the at least one alignment that has the highest alignment score therefrom and store said one of the at least one alignment in said L1-cache 43. Said one of the at least one alignment thus reserved is related to the haplotype.

In one embodiment, for each of said at least one diagonal, the programmable core 41 is configured to assign the diagonal with the diagonal index according to the ascending order of the row of the start cell of the diagonal, the ascending order of the length of the diagonal, and the ascending order of the column of the end cell of the diagonal. The programmable core 41 is configured to designate the series of bins to the X number of rows of the bit matrix H in a manner that every consecutive b number of rows from the start of the rows of the bit matrix H are designated as a respective one of the bins and remaining one(s) of the rows of the bit matrix H are designated as a last one of the bins, where b is a positive integer such as two. The programmable core 41 is configured to build the bin-index table that records the start index and the end index for each of the bins. The programmable core 41 is configured to initialize the bin-index table by assigning, for each of the bins, the positive predetermined value to the start index and the negative predetermined value to the end index. The programmable core 41 is configured to perform initial filling on the bin-index table by, for each of the bins in the bin-index table, updating the start index of the bin with the diagonal index of the first one of said at least one diagonal having the end row that is designated to the bin, and updating the end index of the bin with the diagonal index of the last one of said at least one diagonal having the start row that is designated to the bin. The programmable core 41 is configured to perform left extension on the bin-index table by, for each of the bins having the start index that is equal to the positive predetermined value, updating the start index of the bin with the start index of the closest subsequent one of the bins having the start index that is not equal to positive predetermined value. The programmable core 41 is configured to perform correction on the bin-index table by, for each of the bins having the start index that is greater than the start index of the subsequent one of the bins, updating the start index of the bin with the start index of the subsequent one of the bins. The programmable core 41 is configured to perform right extension on the bin-index table by, for each of the bins having the end index that is equal to the negative predetermined value, updating the end index of the bin with the end index of the closest prior one of the bins having the end index that is not equal to the negative predetermined value. The programmable core 41 is configured to store the bin-index table in the L1-cache 43. For determining the sub-alignment, the programmable core 41 is configured to determine whether the trace region covers one of the diagonals with reference to the bin-index table. In response to the trace region covering one of the diagonals, the programmable core 41 is configured to create the sub-alignment including at least a portion of said one of the diagonals that is located within the trace region. In response to the trace region not covering any one of the diagonals, the programmable core 41 is configured to create the sub-alignment by using the Smith-Waterman algorithm.

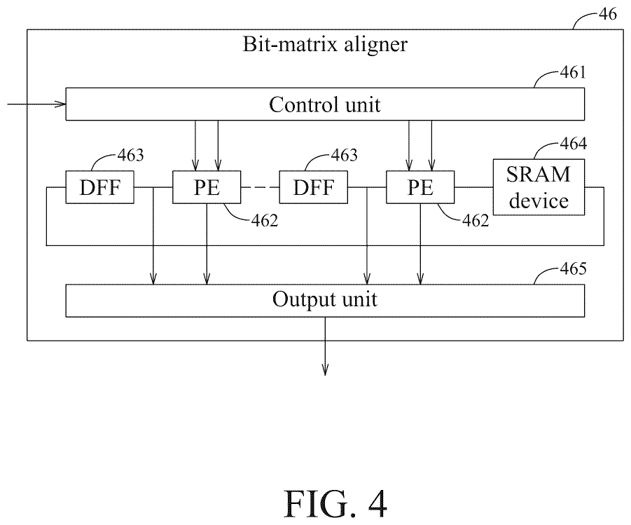

Referring to FIG. 4, the bit-matrix aligner 46 includes a control unit 461, P number of processing pairs, a static random-access memory (SRAM) device 464 and an output unit 465, where P is a positive integer such as four. The control unit 461 is electrically connected to the programmable core 41. The processing pairs are electrically connected in series, and each of the processing pairs includes a processing engine (PE) 462 and a D-flip-flop (DFF) 463. The PE 462 and the DFF 463 of each of the processing pairs are electrically connected to each other. The PE 462 of each of the processing pairs is electrically connected to the control unit 461. The PE 462 of a q^{th} one of the processing pairs is electrically connected to the DFF 463 of a (q+1)^{th} one of the processing pairs, where q is an integer ranging from one to P-1. The SRAM device 464 is electrically connected to the DFF 463 of a first one of the processing pairs and the PE 462 of a P^{th} one of the processing pairs. The output unit 465 is electrically connected to the control unit 461, the L1-cache 43, and to the PE 462 and the DFF 463 of each of the processing pairs.

The control unit 461 is configured to send a bit value of one of the cells of the bit matrix H to the PE 462 of each of the processing pairs. The PE 462 of each of the processing pairs is configured to check the bit value and to update a diagonal length. The DFF 463 of each of the processing pairs is configured to store the bit value and the diagonal length. The SRAM device 464 is configured to store an intermediate diagonal length, and a position of a cell of the bit matrix H where a diagonal terminates. The output unit 465 is configured to receive from the control unit 461 a position of a cell of the bit matrix H that is being scanned, to receive from the PE 462 of one of the processing pairs a termination indicator that has a value of one to indicate a termination of a diagonal, and the diagonal length stored in the DFF 463 of one of the processing pairs.

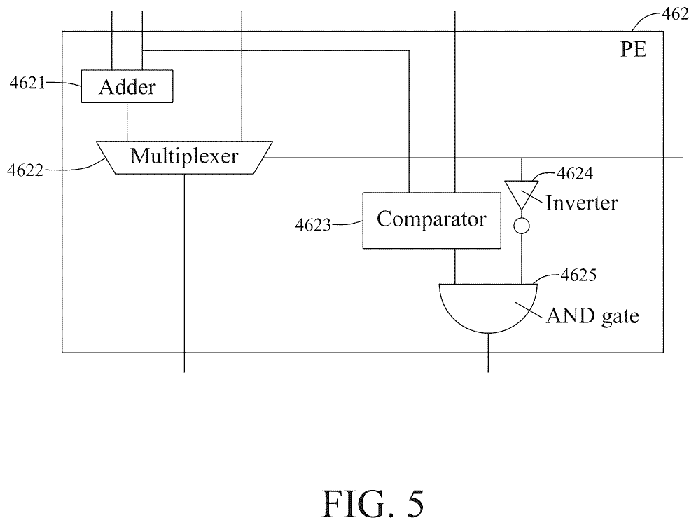

Referring to FIG. 5, the PE 462 of each of the processing pairs includes an adder 4621, a multiplexer 4622, a comparator 4623, an inverter 4624 and an AND gate 4625.

For the PE 462 of the first one of the processing pairs, the adder 4621 has a first input terminal that is configured to receive a value of one, a second input terminal that is electrically connected to the SRAM device 464 for receiving the intermediate diagonal length therefrom, and an output terminal that is configured to output a new diagonal length as a sum of the value of one and the intermediate diagonal length. The multiplexer 4622 has a first input terminal that is electrically connected to the output terminal of the adder 4621 for receiving the new diagonal length therefrom, a second input terminal that is configured to receive a value of zero, a selector terminal that is electrically connected to the control unit 461 for receiving the bit value therefrom, and an output terminal that is electrically connected to the DFF 463 of a second one of the processing pairs, and that is configured to output thereto the new diagonal length in response to the bit value received via the selector terminal being equal to one, and to output thereto the value of zero in response to the bit value received via the selector terminal being equal to zero. The comparator 4623 has a first input terminal that is electrically connected to the SRAM device 464 for receiving the intermediate diagonal length therefrom, a second input terminal that is configured to receive the preset length threshold (e.g., two), and an output terminal that is configured to output a pulse when the intermediate diagonal length is greater than the preset length threshold. The inverter 4624 has an input terminal that is electrically connected to the control unit 461 for receiving the bit value therefrom, and an output terminal for outputting an inverse of the bit value. The AND gate 4625 has a first input terminal that is electrically connected to the output terminal of the comparator 4623 for receiving the pulse therefrom, a second input terminal that is electrically connected to the output terminal of the inverter 4624 for receiving the inverse of the bit value therefrom, and an output terminal that is configured to output the termination indicator in response to the bit value being equal to zero and receive the pulse from the comparator 4623.

For q equal to one of two to (P−1) and the PE 462 of the q^{th} one of said processing pairs, the adder 4621 has a first input terminal that is configured to receive a value of one, a second input terminal that is electrically connected to the DFF 463 of a (q−1)^{th} one of the processing pairs for receiving an old diagonal length therefrom, and an output terminal that is configured to output a new diagonal length as a sum of the value of one and the old diagonal length. The multiplexer 4622 has a first input terminal that is electrically connected to the output terminal of the adder 4621 for receiving the new diagonal length therefrom, a second input terminal that is configured to receive a value of zero, a selector terminal that is electrically connected to the control unit 461 for receiving the bit value therefrom, and an output terminal that is electrically connected to the DFF 463 of the (q+1)^{th} one of the processing pairs, and that is configured to output thereto the new diagonal length in response to the bit value received via the selector terminal equal to one, and to output thereto the value of zero in response to the bit value received via the selector terminal being equal to zero. The comparator 4623 has a first input terminal that is electrically connected to the DFF 463 of the (q−1)^{th} one of the processing pairs for receiving the old diagonal length therefrom, a second input terminal that is configured to receive the preset length threshold, and an output terminal that is configured to output a pulse when the old diagonal length is greater than the preset length threshold. The inverter 4624 has an input terminal that is electrically connected to the control unit 461 for receiving the bit value therefrom, and an output terminal for outputting an inverse of the bit value. The AND gate 4625 has a first input terminal that is electrically connected to the output terminal of the comparator 4623 for receiving the pulse therefrom, a second input terminal that is electrically connected to the output terminal of the inverter 4624 for receiving the inverse of the bit value therefrom, and an output terminal that is configured to output the termination indicator in response to the bit value being equal to zero and receive the pulse from the comparator 4623.

For the PE 462 of the P^{th} one of of the processing pairs, the adder 4621 has a first input terminal that is configured to receive a value of one, a second input terminal that is electrically connected to the DFF 463 of a (P−1)^{th} one of the processing pairs and that is configured to receive an old diagonal length therefrom, and an output terminal for outputting a new diagonal length as a sum of the old diagonal length and the value of one. The multiplexer 4622 has a first input terminal that is electrically connected to the output terminal of the adder 4621 and that is configured to receive therefrom the new diagonal length, a second input terminal that is configured to receive a value of zero, a selector terminal that is electrically connected to the control unit 461 and that is configured to receive the bit value therefrom, and an output terminal that is electrically connected to the SRAM device 464, and that is configured to output thereto the new diagonal length when the bit value received via the selector terminal is one, and to output thereto the value of zero when the bit value received via the selector terminal is zero. The comparator 4623 has a first input terminal that is electrically connected to the DFF 463 of the (P−1)^{th} one of the processing pairs and that is configured to receive the old diagonal length therefrom, a second input terminal that is configured to receive the preset length threshold, and an output terminal that is configured to output a pulse when the old diagonal length is greater than the preset length threshold. The inverter 4624 has an input terminal that is electrically connected to the control unit 461 and that is configured to receive the bit value therefrom, and an output terminal for outputting an inverse of the bit value. The AND gate 4625 has a first input terminal that is electrically connected to the output terminal of the comparator 4623 and that is configured to receive the pulse therefrom, a second input terminal that is electrically connected to the output terminal of the inverter 4624 for receiving the inverse of the bit value therefrom, and an output terminal that is configured to output the termination indicator when the bit value is zero and the comparator 4623 outputs the pulse.

The programmable core 41 is configured to send the input data to the asynchronous MAC unit 45. The vector coprocessor 42 is configured to perform calculations related to match and insertion matrices M, I for the PFA at least based on the input data, and to store results of the calculations related to the match and insertion matrices M, I in the L1-cache 43. The asynchronous MAC unit 45 is configured to perform calculations related to a deletion matrix D for the PFA at least based on the input data, and to store results of the calculations related to the deletion matrix D in the L1-cache 43. It is worth to note that the vector coprocessor 42 and the asynchronous MAC unit 45 are configured to perform the calculations in parallel. The vector coprocessor 42 is configured to calculate read-haplotype likelihoods based on the match and insertion matrices M, I stored in the L1-cache 43, and to send the read-haplotype likelihoods to the programmable core 41 to enable the programmable core 41 to store the read-haplotype likelihoods in the L1-cache 43.

Specifically, each of the match matrix M, the insertion matrix I and the deletion matrix D is an |a|×|b| matrix, where |a| is a number of nucleotides of a haplotype, |b| is a number of nucleotides of one of the overlapping reads, and each of |a| and |b| is a positive integer. A cell M(i,j) of the match matrix M has a value M_{i,j}, a cell I(i,j) of the insertion matrix I has a value I_{i,j}, a cell D(i,j) of the deletion matrix D has a value D_{i,j}, where i is a positive integer ranging from one to |a|, and j is a positive integer ranging from one to |b|.

The vector coprocessor 42 is configured to calculate the value M_{i,j} of the cell M(i,j) of the match matrix M based on:

$${M}_{i , j} = p ( {a}_{i} , {b}_{j} ) ( {M}_{i - 1 , j - 1} {α}_{M M i} + {I}_{i - 1 , j - 1} {α}_{IMi} + {D}_{i - 1 , j - 1} {α}_{D M i} ) ,$$

where p(aᵢ, bⱼ) represents a match/mismatch score between an i^{th} one of the a number of nucleotides of the haplotype and a j^{th} one of the |b| number of nucleotides of the one of the overlapping reads, α_{efg} represents a transition probability for a quality score of a g^{th} one of the |a| number of nucleotides of the haplotype to transit from an e state among match (M), insertion (I) and deletion (D) states to an f state among the match (M), insertion (I) and deletion (D) states.

The vector coprocessor 42 is configured to calculate the value I_{i,j} of the cell I(i,j) of the insertion matrix I based on:

$${I}_{i , j} = {I}_{i - 1 , j} {α}_{MIi} + {I}_{i - 1 , j} {α}_{IIi} .$$

The asynchronous MAC unit 45 is configured to calculate the value D_{i,j} of the cell D(i,j) of the deletion matrix D based on:

$${D}_{i , j} = {M}_{i , j - 1} {α}_{M D i} + {D}_{i , j - 1} {α}_{D D i} .$$

The vector coprocessor 42 is configured to calculate a j^{th} one of the read-haplotype likelihoods as M_{|a|,j}+I_{|a|,j} for 1≤j≤|b|.

In a scenario where each of the match matrix M, the insertion matrix I and the deletion matrix D has two rows and two columns, for two tasks of calculating values M_{u,v,w} respectively of cells M(v,w) of the match matrix M, values I_{u,v,w} respectively of cells I(v,w) of the insertion matrix I, and values D_{u,v,w} respectively of cells D(v,w) of the deletion matrix D, wherein u is an index of the two tasks, and each of u, v and w is a positive integer ranging from one to two, the vector coprocessor 42 and the asynchronous MAC unit 45 are configured to cooperatively perform the two tasks in a manner of row-major inter-task PFA scheme as shown in FIG. 10 (calculations of matrices are performed from left to right in order) and delineated below.

Firstly, the vector coprocessor 42 is configured to calculate values M_{1,1,1} and M_{1,1,2} respectively of cells M(1,1) and M(1,2) of the match matrix M for a first one of the two tasks.

Secondly, the vector coprocessor 42 is configured to calculate values I_{1,1,1} and I_{1,1,2} respectively of cells I(1,1) and I(1,2) of the insertion matrix I for the first one of the two tasks.

Thirdly, the vector coprocessor 42 is configured to calculate values M_{2,1,1} and M_{2,1,2} respectively of cells M(1,1) and M(1,2) of the match matrix M for a second one of the two tasks, and at the same time, the asynchronous MAC unit 45 is configured to calculate a value D_{1,1,1} of a cell D(1,1) of the deletion matrix D for the first one of the two tasks.

Fourthly, the vector coprocessor 42 is configured to calculate values I_{2,1,1} and I_{2,1,2} respectively of cells I(1,1) and I(1,2) of the insertion matrix I for the second one of the two tasks, and at the same time, the asynchronous MAC unit 45 is configured to calculate a value D_{1,1,2} of a cell D(1,2) of the deletion matrix D for the first one of the two tasks.

Fifthly, the vector coprocessor 42 is configured to calculate values M_{1,2,1} and M_{1,2,2} respectively of cells M(2,1) and M(2,2) of the match matrix M for the second one of the two tasks, and at the same time, the asynchronous MAC unit 45 is configured to calculate a value D_{2,1,1} of a cell D(1,1) of the deletion matrix D for the second one of the two tasks.

Sixthly, the vector coprocessor 42 is configured to calculate values I_{1,2,1} and I_{1,2,2} respectively of cells I(2,1) and I(2,2) of the insertion matrix I for the second one of the two tasks, and at the same time, the asynchronous MAC unit 45 is configured to calculate a value D_{2,1,2} of a cell D(1,2) of the deletion matrix D for the second one of the two tasks.

Seventhly, the vector coprocessor 42 is configured to calculate values M_{2,2,1} and M_{2,2,2} respectively of cells M(2,1) and M(2,2) of the match matrix M for the second one of the two tasks, and at the same time, the asynchronous MAC unit 45 is configured to calculate a value D_{1,2,1} of a cell D(2,1) of the deletion matrix D for the first one of the two tasks.

Eighthly, the vector coprocessor 42 is configured to calculate values I_{2,2,1} and I_{2,2,2} respectively of cells I(2,1) and I(2,2) of the insertion matrix I for the second one of the two tasks, and at the same time, the asynchronous MAC unit 45 is configured to calculate a value D_{1,2,2} of a cell D(2,2) of the deletion matrix D for the first one of the two tasks.

Ninthly, the asynchronous MAC unit 45 is configured to calculate a value D_{2,2,1} of a cell D(2,1) of the deletion matrix D for the second one of the two tasks.

Tenthly, the asynchronous MAC unit 45 is configured to calculate a value D_{2,2,2} of a cell D(2,2) of the deletion matrix D for the second one of the two tasks.

The system according to the disclosure may be exemplarily implemented by using a semiconductor chip fabricated with 40 nm process technology, and details of hardware resource requirements are listed in Table 10 below. The RISC-V programmable core operates at 1500 MHz and consumes relatively low power of 2.10 mW. The overall of the first processing devices 3 occupies an area of 113.27 mm² and consumes power of 41.89 W. A total number of the second processing devices 4 is sixteen, and the overall of the second processing devices 4 occupies an area of 41.12 mm² and consumes power of 12.27 W.

TABLE 10

| Component | Area (μm²) | Power(mW) |
|---|---|---|
| RISC-V programmable | 19786.1 | 2.10 |
| core | | |
| 8-lane vector | 15000 | 2.85 |
| coprocessor | | |
| Asynchronous MAC unit | 19066 | 26.4 |
| 64 KB L1-cache | 125829 | 42.99 |
| 256 KB L2-cache | 503316 | 171.97 |
| Bit-matrix aligner | 5690 | 1.05 |
| (without SRAM) | | |
| Bit-matrix aligner | 5420 | 2.29 |
| (with 4 KB SRAM) | | |

The system according to the disclosure offers parallelism at multiple levels. Firstly, the CPU 1 is implemented to use multithreading and operates in a coarse-grained parallel manner to identify the active region. Secondly, the first processing devices 3 and the second processing devices 4 operate in a coarse-grained parallel manner to execute tasks being assigned, such as tasks of DBG generation and traversal, local alignment and PFA. Thirdly, the vector coprocessors 32, 42 respectively of the first processing devices 3 and the second processing devices 4, the asynchronous MAC units 45 respectively of the second processing devices 4, and the bit-matrix aligners 46 respectively of the second processing devices 4 operate in a fine-grained parallel manner to fully exploit hardware resources, and parallel operations thereof are dependent upon data layout required to support regular memory accesses.

Tables 11 and 12 cooperatively show pseudocode for a bit-matrix algorithm (BMA), where utilization of the bin-index table has been incorporated.

TABLE 11
/* bmAlign: Calculate Bit-Matrix Alignment between a reference
(R) and a query string (Q), while recording diagonals longer
than thresh, using scoring parameters in params.scores */

| | |
|---|---|
| 1: | bv ← calculateBitVectors(R) |
| 2: | diagData, longDiagIdxs ← calcDiags(bv, Q, thresh, n) |
| 3: | BIndexes ← generateBIndexes(diagData) |
| 4: | maxScore ← 0 |
| 5: | alignResult ← emptyString( ) |
| 6: | for firstIdx ← 0 to length(longDiagIdxs)-1 do |
| 7: | Params.traceBounds ← 0, 0, length(Q)-1, length(R)-1 |
| 8: | consolidatedAln, consolidatedScore ← empty( ) |
| 9: | consolidatedAln, consolidatedScore ← trace(Q, R, firstIdx, diagData, |
| | params, consolidatedAln, consolidatedScore) |
| 10: | if consolidatedScore > maxScore then |
| 11: | maxScore ← consolidatedScore |
| 12: | alignResult ← consolidatedAln |
| 13: | endif |
| 14: | endfor |
| 15: | return maxScore, alignResult |

TABLE 12
/* trace: Perform traceback using diagonals data diagData, starting with
diagonal at array index firstIdx within bounds param.traceBounds, and
update the previous partial alignment results currAln, currScore */

| | |
|---|---|
| 1: | if isInvalidBounds(params.traceBounds) then |
| 2: | return currAln, currScore |
| 3: | endif |
| 4: | tracedScore ← currScore + diagData[firstIdx].length * params.scores.M |
| 5: | traceAln ← updateAln(currAln, string(diagData[firstIdx].length) + ‘M’) |
| 6: | longestDiagIdxs ← findLongestDiagsInBounds(diagData, Bindexes, |
| | params.traceBounds) |
| 7: | i ← 0 |
| 8: | while isFullQueryNotCovered(tracedAln) and i < length(longestDiagIdxs) |
| | do |
| 9: | lParams.traceBounds ← findLeftTraceBounds(tracedAln, |
| | diagData[longestDiagIdxs[i]]) |
| 10: | rParams.traceBounds ← findRightTraceBounds(tracedAln, |
| | diagData[longestDiagIdxs[i]]) |
| 11: | lTracedAln, leftTracedScore ← trace(diagData, longestDiagIdxs[i], |
| | lParams, tracedAln, tracedScore, “left”) |
| 12: | rTracedAln, rightTracedScore ← trace(diagData, longestDiagIdxs[i], |
| | rParams, tracedAln, tracedScore, “right”) |
| 13: | consolidatedAln, consolidatedScore ← consolidateResults(tracedAln, |
| | tracedScore, lTracedAln, lTracedScore, rTracedAln, rTracedScore) |
| 14: | if consolidatedScore > tracedScore then |
| 15: | tracedAln, tracedScore ← consolidatedAln, consolidatedScore |
| 16: | endif |
| 17: | endwhile |
| 18: | if length(longestDiagIdxs) == 0 then |
| 19: | tracedAln, tracedScore ← naiveSW (Q, R, params) |
| 20: | consolidatedAln, consolidatedScore ← consolidateResults(currAln, |
| | currScore, tracedAln, tracedScore) |
| 21: | if consolidatedScore > tracedScore then |
| 22: | tracedAln, tracedScore ← consolidatedAln, consolidatedScore |
| 23: | endif |
| 24: | endif |
| 25: | return tracedAln, tracedScore |

To sum up, the method of variant calling according to the disclosure implements the local alignment by using BMA, where the bit matrix H is utilized to find the alignment that is related to the haplotype. To enhance efficiency of performing the method, the bin-index table is incorporated into the method to facilitate looking up the diagonal in the bit matrix H. Compared to GATK Haplotypecaller, the method according to the disclosure requires less hardware resources but attains higher efficiency. Moreover, single-instruction-multiple-data-optimized (SIMD-optimized) distributed processing is adopted to facilitate variant calling by using the system according to the disclosure. In particular, the first processing devices 3 of the distributed processing module 2 of the system are utilized to perform DBG generation and traversal, and the second processing devices 4 of the distributed processing module 2 are utilized to perform the local alignment and the PFA. Furthermore, the CPU 1 of the system is capable of dynamically dividing the second processing devices 4 into clusters of various sizes, and distributing tasks to the clusters according to the sizes respectively of the clusters and scales respectively of the tasks. Overall, the system according to the disclosure offers parallelism at a variety of levels, and may be capable of enhancing efficiency of completing tasks for variant calling.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for performing local alignment based on a query sequence of deoxyribonucleic acid (DNA) and a reference sequence of DNA, the method comprising:
obtaining a bit matrix H that has an X number of rows each of which is related to a nucleotide of the query sequence of DNA, and a Y number of columns each of which is related to a nucleotide of the reference sequence of DNA, a cell H(m,n) of the bit matrix H having a value of one when an m^{th} one of nucleotides of the query sequence of DNA is identical to an n^{th} one of nucleotides of the reference sequence of DNA, and having a value of zero when the m^{th} one of nucleotides of the query sequence of DNA is different from the n^{th} one of nucleotides of the reference sequence of DNA, where X and Y are each a positive integer, m is an integer ranging from zero to (X−1), and n is an integer ranging from zero to (Y−1);
determining, based on the bit matrix H, at least one diagonal that contains d number of consecutive cells from H(m,n) to H(m+d−1,n+d−1) of the bit matrix H, where d is a positive integer greater than a preset length threshold and represents a length of said at least one diagonal;
for each of said at least one diagonal that contains a start cell H(m,n) and an end cell H(m+d−1,n+d−1),
calculating an initial score for the diagonal according to a predetermined scoring scheme of sequence alignment,
determining at least one trace region in the bit matrix H, said at least one trace region being defined by trace bounds that include one of a first pair of cells of the bit matrix H and a second pair of cells of the bit matrix H, the first pair of cells including the start cell H(m,n) of the diagonal and a start cell H(0,0) of the bit matrix H, the second pair of cells including the end cell H(m+d−1,n+d−1) of the diagonal and an end cell H(X−1,Y−1) of the bit matrix H,
for each of said at least one trace region, determining a sub-alignment that is one of paths passing through cells in the trace region from an upper-left corner to a lower-right corner of the trace region, and that has a highest partial score among all of the paths according to the predetermined scoring scheme of sequence alignment,
consolidating the diagonal and the sub-alignment respectively of said at least one trace region to obtain an alignment, and
obtaining an alignment score based on the initial score and the partial score respectively of the sub-alignment respectively of said at least one trace region; and
among each of the at least one alignment thus determined respectively for each of said at least one diagonal, reserving one of the at least one alignment that has the highest alignment score therefrom, said one of the at least one alignment thus reserved being related to a haplotype.

2. The method as claimed in claim 1, further comprising:
for each of said at least one diagonal, assigning the diagonal with a diagonal index that is an integer according to an ascending order of a row of the start cell of the diagonal, an ascending order of the length of the diagonal, and an ascending order of a column of the end cell of the diagonal;
designating a series of bins to the X number of rows of the bit matrix H in a manner that every consecutive b number of rows from the start of the rows of the bit matrix H are designated as a respective one of the bins and remaining one(s) of the rows of the bit matrix H are designated as a last one of the bins, where b is a positive integer;
building a bin-index table that records a start index and an end index for each of the bins;
initializing the bin-index table by assigning, for each of the bins, a positive predetermined value to the start index and a negative predetermined value to the end index;
performing initial filling on the bin-index table by, for each of the bins in the bin-index table, updating the start index of the bin with the diagonal index of a first one of said at least one diagonal having the end row that is designated to the bin, and updating the end index of the bin with the diagonal index of a last one of said at least one diagonal having the start row that is designated to the bin;
performing left extension on the bin-index table by, for each of the bins having the start index that is equal to the positive predetermined value, updating the start index of the bin with the start index of a closest subsequent one of the bins having the start index that is not equal to the positive predetermined value;
performing correction on the bin-index table by, for each of the bins having the start index that is greater than the start index of a subsequent one of the bins, updating the start index of the bin with the start index of the subsequent one of the bins; and
performing right extension on the bin-index table by, for each of the bins having the end index that is equal to the negative predetermined value, updating the end index of the bin with the end index of a closest prior one of the bins having the end index that is not equal to the negative predetermined value,
wherein determining a sub-alignment includes
determining whether the trace region covers one of said at least one diagonal with reference to the bin-index table,
in response to the trace region covering one of said at least one diagonals, creating the sub-alignment including at least a portion of said one of said at least one diagonal that is located within the trace region, and
in response to the trace region not covering any one of said at least one diagonal, creating the sub-alignment by using a Smith-Waterman algorithm.

3. A method of variant calling, comprising:
identifying an active region on a reference sequence of deoxyribonucleic acid (DNA) according to a plurality of overlapping reads that are obtained in DNA sequencing, each of the overlapping reads representing a sequence that overlaps with the active region of the reference sequence of DNA;
generating a de Brujin graph (DBG) based on the overlapping reads and the active region of the reference sequence of DNA;
performing sequential traversal on the DBG to determine a query sequence of DNA;
obtaining a bit matrix H that has an X number of rows each of which is related to a nucleotide of the query sequence of DNA, and a Y number of columns each of which is related to a nucleotide of a reference sequence of DNA, a cell H(m,n) of the bit matrix H having a value of one when an m^{th} one of nucleotides of the query sequence of DNA is identical to an n^{th} one of nucleotides of the reference sequence of DNA, and having a value of zero when the m^{th} one of nucleotides of the query sequence of DNA is different from the n^{th} one of nucleotides of the reference sequence of DNA, where X and Y are each a positive integer, m is an integer ranging from zero to (X−1), and n is an integer ranging from zero to (Y−1);
determining, based on the bit matrix H, at least one diagonal that contains d number of consecutive cells from H(m,n) to H(m+d−1,n+d−1) of the bit matrix H, where d is a positive integer greater than a preset length threshold and represents a length of said at least one diagonal;
for each of said at least one diagonal that contains a start cell H(m,n) and an end cell H(m+d−1,n+d−1),
calculating an initial score for the diagonal according to a predetermined scoring scheme of sequence alignment,
determining at least one trace region in the bit matrix H, said at least one trace region being defined by trace bounds that include one of a first pair of cells of the bit matrix H and a second pair of cells of the bit matrix H, the first pair of cells including the start cell H(m,n) of the diagonal and a start cell H(0,0) of the bit matrix H, the second pair of cells including the end cell H(m+d−1,n+d−1) of the diagonal and an end cell H(X−1,Y−1) of the bit matrix H,
for each of said at least one trace region, determining a sub-alignment that is one of paths passing through cells in the trace region from an upper-left corner to a lower-right corner of the trace region, and that has a highest partial score among all of the paths according to the predetermined scoring scheme of sequence alignment,
consolidating the diagonal and the sub-alignment respectively of said at least one trace region to obtain an alignment, and
obtaining an alignment score based on the initial score and the partial score respectively of the sub-alignment respectively of said at least one trace region;
among each of the at least one alignment thus determined respectively for each of said at least one diagonal, reserving one of the at least one alignment that has the highest alignment score therefrom, said one of the at least one alignment thus reserved being related to a haplotype;
determining read-haplotype likelihoods by using a pair hidden Markov model (pair-HMM) forward algorithm (PFA) based on the haplotype and the overlapping reads; and
determining a genotype by using Bayes' Theorem based on the read-haplotype likelihoods thus determined.

4. A processing device for facilitating variant calling, said processing device being adapted to be electrically connected to a central processing unit (CPU), and comprising:
a programmable core;
a vector coprocessor electrically connected to said programmable core; and
an L1-cache electrically connected to said programmable core and said vector coprocessor, and configured to store input data offloaded by the CPU, the input data containing information about an active region of a reference sequence of deoxyribonucleic acid (DNA) and a plurality of overlapping reads that are obtained in DNA sequencing, each of the overlapping reads representing a sequence that overlaps with the active region of the reference sequence of DNA,
wherein said programmable core is configured to read the input data from said L1-cache, and to send the input data to said vector coprocessor,
wherein said vector coprocessor is configured to perform k-mer hashing and unique k-mer detection based on the input data, and to store results respectively of k-mer hashing and unique k-mer detection in said L1-cache, where k is a positive integer, and
wherein said programmable core is configured to read the results of k-mer hashing and unique k-mer detection from said L1-cache, to generate a de Brujin graph (DBG) based on the results of k-mer hashing and unique k-mer detection, to perform sequential traversal on the DBG to determine a query sequence of DNA, and to store information about the query sequence of DNA in said L1-cache.

5. The processing device as claimed in claim 4, wherein said vector coprocessor is further configured to, for each of the overlapping reads:
select a plurality of sub-sequences from a sequence represented by the overlapping read using a sliding window of size three, each of the sub-sequences containing three of consecutive nucleotides in the sequence;
for each of sub-sequences, pad the sub-sequence with an additional predetermined nucleotide before the three of the consecutive nucleotides to generate a 4-mer;
encode each of the 4-mers that are generated respectively for the sub-sequences into an eight-bit binary sequence, the eight-bit binary sequence representing a hash value that includes a pair of numbers, a set of first four bits of the eight-bit binary sequence representing one of the pair of numbers of the hash value, a set of last four bits of the eight-bit binary sequence representing another of the pair of numbers of the hash value;
build a hash table that has a plurality of rows each having a plurality of sectors;
for each of the eight-bit binary sequences corresponding respectively to the 4-mers, assign said another of the pair of numbers of the hash value represented by the eight-bit binary sequence to one of the sectors of the hash table, and record k-mer data related to the 4-mer that corresponds to the eight-bit binary sequence in the one of the sectors of the hash table, the k-mer data containing information about the 4-mer and a nucleotide to which the three number of consecutive nucleotides corresponding to the 4-mer are to transit.

6. The processing device as claimed in claim 4, further comprising an asynchronous medium access control (MAC) unit electrically connected to said programmable core and said L1-cache,
wherein said programmable core is configured to send the input data to said asynchronous MAC unit,
wherein said vector coprocessor is configured to perform calculations related to match and insertion matrices M, I for a pair hidden Markov model (pair-HMM) forward algorithm (PFA) at least based on the input data, and to store results of the calculations related to the match and insertion matrices M, I in said L1-cache,
wherein said asynchronous MAC unit is configured to perform calculations related to a deletion matrix D for the PFA at least based on the input data, and to store results of the calculations related to the deletion matrix D in said L1-cache,
wherein said vector coprocessor and said asynchronous MAC unit are configured to perform the calculations in parallel,
wherein said vector coprocessor is configured to calculate read-haplotype likelihoods based on the match and insertion matrices M, I stored in said L1-cache, and to send the read-haplotype likelihoods to said programmable core to enable said programmable core to store the read-haplotype likelihoods in said L1-cache.

7. The processing device as claimed in claim 6, wherein:
each of the match matrix M, the insertion matrix I and the deletion matrix D is an |a|×|b| matrix, |a| being a number of nucleotides of a haplotype, |b| being a number of nucleotides of one of the overlapping reads, where a cell M(i,j) of the match matrix M has a value M_{i,j}, a cell I(i,j) of the insertion matrix I has a value I_{i,j}, a cell D(i,j) of the deletion matrix D has a value D_{i,j}, each of |a| and |b| is a positive integer, i is a positive integer ranging from one to |a|, and j is a positive integer ranging from one to |b|;
said vector coprocessor is configured to calculate the value M_{i,j} of the cell M(i,j) of the match matrix M based on:
$${M}_{i , j} = p ( {a}_{i} , {b}_{j} ) ( {M}_{i - 1 , j - 1} {α}_{M M i} + {I}_{i - 1 , j - 1} {α}_{IMi} + {D}_{i - 1 , j - 1} {α}_{D M i} ) ,$$
where p(aᵢ, bⱼ) represents a match/mismatch score between an i^{th} one of the |a| number of nucleotides of the haplotype and an j^{th} one of the |b| number of nucleotides of the one of the overlapping reads, α_{efg} represents a transition probability for a quality score of a g^{th} one of the |a| number of nucleotides of the haplotype to transit from an e state among match, insertion and deletion states to an f state among the match, insertion and deletion states;
said vector coprocessor is configured to calculate the value I_{i,j} of the cell I(i,j) of the insertion matrix I based on:
$${I}_{i , j} = {I}_{i - 1 , j} {α}_{MIi} + {I}_{i - 1 , j} {α}_{IIi} ;$$
said asynchronous MAC unit is configured to calculate the value D_{i,j} of the cell D(i,j) of the deletion matrix D based on:
$${D}_{i , j} = {M}_{i , j - 1} {α}_{M D i} + {D}_{i , j - 1} {α}_{D D i} ;$$
and
said vector coprocessor is configured to calculate a j^{th} one of the read-haplotype likelihoods as M_{|a|,j}+I_{|a|,j} for 1≤j≤|b|.

8. The processing device as claimed in claim 6, wherein each of the match matrix M, the insertion matrix I and the deletion matrix D has two rows and two columns,
wherein, two tasks of calculating values M_{u,v,w} respectively of cells M(v,w) of the match matrix M, values I_{u,v,w} respectively of cells I(v,w) of the insertion matrix I, and values D_{u,v,w} respectively of cells D(v,w) of the deletion matrix D, where u is an index of the two tasks, and each of u, v and w is a positive integer ranging from one to two,
firstly, said vector coprocessor is configured to calculate values M_{1,1,1} and M_{1,1,2} respectively of cells M(1,1) and M(1,2) of the match matrix M for a first one of the two tasks,
secondly, said vector coprocessor is configured to calculate values I_{1,1,1} and I_{1,1,2} respectively of cells I(1,1) and I(1,2) of the insertion matrix I for the first one of the two tasks,
thirdly, said vector coprocessor is configured to calculate values M_{2,1,1} and M_{2,1,2} respectively of cells M(1,1) and M(1,2) of the match matrix M for a second one of the two tasks, and at the same time, said asynchronous MAC unit is configured to calculate a value D_{1,1,1} of a cell D(1,1) of the deletion matrix D for the first one of the two tasks,
fourthly, said vector coprocessor is configured to calculate values I_{2,1,1} and I_{2,1,2} respectively of cells I(1,1) and I(1,2) of the insertion matrix I for the second one of the two tasks, and at the same time, said asynchronous MAC unit is configured to calculate a value D_{1,1,2} of a cell D(1,2) of the deletion matrix D for the first one of the two tasks,
fifthly, said vector coprocessor is configured to calculate values M_{1,2,1} and M_{1,2,2} respectively of cells M(2,1) and M(2,2) of the match matrix M for the second one of the two tasks, and at the same time, said asynchronous MAC unit is configured to calculate a value D_{2,1,1} of a cell D(1,1) of the deletion matrix D for the second one of the two tasks,
sixthly, said vector coprocessor is configured to calculate values I_{1,2,1} and I_{1,2,2} respectively of cells I(2,1) and I(2,2) of the insertion matrix I for the second one of the two tasks, and at the same time, said asynchronous MAC unit is configured to calculate a value D_{2,1,2} of a cell D(1,2) of the deletion matrix D for the second one of the two tasks,
seventhly, said vector coprocessor is configured to calculate values M_{2,2,1} and M_{2,2,2} respectively of cells M(2,1) and M(2,2) of the match matrix M for the second one of the two tasks, and at the same time, said asynchronous MAC unit is configured to calculate a value D_{1,2,1} of a cell D(2,1) of the deletion matrix D for the first one of the two tasks,
eighthly, said vector coprocessor is configured to calculate values I_{2,2,1} and I_{2,2,2} respectively of cells I(2,1) and I(2,2) of the insertion matrix I for the second one of the two tasks, and at the same time, said asynchronous MAC unit is configured to calculate a value D_{1,2,2} of a cell D(2,2) of the deletion matrix D for the first one of the two tasks,
ninthly, said asynchronous MAC unit is configured to calculate a value D_{2,2,1} of a cell D(2,1) of the deletion matrix D for the second one of the two tasks, and
tenthly, said asynchronous MAC unit is configured to calculate a value D_{2,2,2} of a cell D(2,2) of the deletion matrix D for the second one of the two tasks.

9. The processing device as claimed in claim 4, further comprising:
a bit-matrix aligner electrically connected to said programmable core and said L1-cache,
wherein said programmable core is configured to generate bit vectors based on the reference sequence of DNA contained in the input data, and to send the bit vectors to said bit-matrix aligner;
wherein said bit-matrix aligner is configured to generate, based on the bit vectors and the query sequence of DNA, a bit matrix H that has an X number of rows each of which is related to a nucleotide of the query sequence of DNA, and a Y number of columns each of which is related to a nucleotide of a reference sequence of DNA, a cell H(m,n) of the bit matrix H having a value of one when an m^{th} one of nucleotides of the query sequence of DNA is identical to an n^{th} one of nucleotides of the reference sequence of DNA, and having a value of zero when the m^{th} one of nucleotides of the query sequence of DNA is different from the n^{th} one of nucleotides of the reference sequence of DNA, where X and Y are each a positive integer, m is an integer ranging from zero to (X−1), and n is an integer ranging from zero to (Y−1),
wherein said bit-matrix aligner is further configured to determine at least one diagonal of the bit matrix H, and to store said at least one diagonal in said L1-cache, said at least one diagonal containing d number of consecutive cells from H(m,n) to H(m+d−1,n+d−1) of the bit matrix H, where d is a positive integer greater than a preset length threshold and represents a length of said at least one diagonal,
wherein said programmable core is configured to, for each of said at least one diagonal that contains a start cell H(m,n) and an end cell H(m+d−1,n+d−1),
calculate an initial score for the diagonal according to a predetermined scoring scheme of sequence alignment,
determine at least one trace region in the bit matrix H, said at least one trace region being defined by trace bounds that include one of a first pair of cells of the bit matrix H and a second pair of cells of the bit matrix H, the first pair of cells including the start cell H(m,n) of the diagonal and a start cell H(0,0) of the bit matrix H, the second pair of cells including the end cell H(m+d−1,n+d−1) of the diagonal and an end cell H(X−1,Y−1) of the bit matrix H,
for each of said at least one trace region, determine a sub-alignment that is one of paths passing through cells in the trace region from an upper-left corner to a lower-right corner of the trace region, and that has a highest partial score among all of the paths according to the predetermined scoring scheme of sequence alignment,
consolidate the diagonal and the sub-alignment respectively of said at least one trace region to obtain an alignment, and
obtain an alignment score based on the initial score and the partial score respectively of the sub-alignment respectively of said at least one trace region,
wherein said programmable core is configured to, among each of the at least one alignment thus determined respectively for each of said at least one diagonal, reserve one of the at least one alignment that has the highest alignment score therefrom and store said one of the at least one alignment in said L1-cache, said one of the at least one alignment thus reserved being related to a haplotype.

10. The processing device as claimed in claim 9, wherein said programmable core is configured to:
for each of said at least one diagonal, assign the diagonal with a diagonal index that is an integer according to an ascending order of a row of the start cell of the diagonal, an ascending order of the length of the diagonal, and an ascending order of a column of the end cell of the diagonal;
designate a series of bins to the X number of rows of the bit matrix H in a manner that every consecutive b number of rows from the start of the rows of the bit matrix H are designated as a respective one of the bins and remaining one(s) of the rows of the bit matrix H are designated as a last one of the bins, where b is a positive integer;
build a bin-index table that records a start index and an end index for each of the bins;
initialize the bin-index table by assigning, for each of the bins, a positive predetermined value to the start index and a negative predetermined value to the end index;
perform initial filling on the bin-index table by, for each of the bins in the bin-index table, updating the start index of the bin with the diagonal index of a first one of said at least one diagonal having the end row that is designated to the bin, and updating the end index of the bin with the diagonal index of a last one of said at least one diagonal having the start row that is designated to the bin;
perform left extension on the bin-index table by, for each of the bins having the start index that is equal to the positive predetermined value, updating the start index of the bin with the start index of a closest subsequent one of the bins having the start index that is not equal to positive predetermined value;
perform correction on the bin-index table by, for each of the bins having the start index that is greater than the start index of a subsequent one of the bins, updating the start index of the bin with the start index of the subsequent one of the bins;
perform right extension on the bin-index table by, for each of the bins having the end index that is equal to the negative predetermined value, updating the end index of the bin with the end index of a closest prior one of the bins having the end index that is not equal to the negative predetermined value; and
store the bin-index table in said L1-cache,
wherein said programmable core determining the sub-alignment is to
determine whether the trace region covers one of the diagonals with reference to the bin-index table,
in response to the trace region covering one of the diagonals, create the sub-alignment including at least a portion of said one of the diagonals that is located within the trace region, and
in response to the trace region not covering any one of the diagonals, create the sub-alignment by using a Smith-Waterman algorithm.

11. The processing device as claimed in claim 9, wherein said bit-matrix aligner including:
a control unit electrically connected to said programmable core;
P number of processing pairs electrically connected in series, each of said processing pairs including a processing engine (PE) and a D-flip-flop (DFF), P being a positive integer, said PE and said DFF of each of said processing pairs being electrically connected to each other, said PE of each of said processing pairs being electrically connected to said control unit, said PE of a q^{th} one of said processing pairs being electrically connected to said DFF of a (q+1)^{th} one of said processing pairs, q being an integer ranging from one to P−1;
a static random-access memory (SRAM) device electrically connected to said DFF of a first one of said processing pairs and said PE of a P^{th} one of said processing pairs; and
an output unit electrically connected to said control unit, said L1-cache, and to said PE and said DFF of each of said processing pairs,
wherein said control unit is configured to send a bit value of one of the cells of the bit matrix H to said PE of each of said processing pairs,
wherein said PE of each of said processing pairs is configured to check the bit value and to update a diagonal length,
wherein said DFF of each of said processing pairs is configured to store the bit value and the diagonal length,
wherein said SRAM device is configured to store an intermediate diagonal length, and a position of a cell of the bit matrix H where a diagonal terminates, and
wherein said output unit is configured to receive from said control unit a position of a cell of the bit matrix H that is being scanned, to receive from said PE of one of said processing pairs a termination indicator that has a value of one to indicate a termination of a diagonal, and the diagonal length stored in said DFF of one of said processing pairs.

12. The processing device as claimed in claim 11, wherein said PE of the first one of said processing pairs includes:
an adder having a first input terminal that is configured to receive a value of one, a second input terminal that is electrically connected to said SRAM device for receiving the intermediate diagonal length therefrom, and an output terminal that is configured to output a new diagonal length as a sum of the value of one and the intermediate diagonal length;
a multiplexer having a first input terminal that is electrically connected to said output terminal of said adder for receiving the new diagonal length therefrom, a second input terminal that is configured to receive a value of zero, a selector terminal that is electrically connected to said control unit for receiving the bit value therefrom, and an output terminal that is electrically connected to said DFF of a second one of said processing pairs, and that is configured to output thereto the new diagonal length in response to the bit value received via the selector terminal equal to one, and to output thereto the value of zero in response to the bit value received via the selector terminal equal to zero;
a comparator having a first input terminal that is electrically connected to said SRAM device for receiving the intermediate diagonal length therefrom, a second input terminal that is configured to receive the preset length threshold, and an output terminal that is configured to output a pulse when the intermediate diagonal length is greater than the preset length threshold;
an inverter having an input terminal that is electrically connected to said control unit for receiving the bit value therefrom, and an output terminal for outputting an inverse of the bit value; and
an AND gate having a first input terminal that is electrically connected to said output terminal of said comparator for receiving the pulse therefrom, a second input terminal that is electrically connected to said output terminal of said inverter for receiving the inverse of the bit value therefrom, and an output terminal that is configured to output the termination indicator in response to the bit value being equal to zero and to receive the pulse from said comparator.

13. The processing device as claimed in claim 11, wherein for q equal to one of two to (P−1), said PE of the q^{th} one of said processing pairs includes:
an adder having a first input terminal that is configured to receive a value of one, a second input terminal that is electrically connected to said DFF of a (q−1)^{th} one of said processing pairs for receiving an old diagonal length therefrom, and an output terminal that is configured to output a new diagonal length as a sum of the value of one and the old diagonal length;
a multiplexer having a first input terminal that is electrically connected to said output terminal of said adder for receiving the new diagonal length therefrom, a second input terminal that is configured to receive a value of zero, a selector terminal that is electrically connected to said control unit for receiving the bit value therefrom, and an output terminal that is electrically connected to said DFF of the (q+1)^{th} one of said processing pairs, and that is configured to output thereto the new diagonal length in response to the bit value received via the selector terminal equal to one, and to output thereto the value of zero in response to the bit value received via the selector terminal being equal to zero;
a comparator having a first input terminal that is electrically connected to said DFF of the (q−1)^{th} one of said processing pairs for receiving the old diagonal length therefrom, a second input terminal that is configured to receive the preset length threshold, and an output terminal that is configured to output a pulse when the old diagonal length is greater than the preset length threshold;
an inverter having an input terminal that is electrically connected to said control unit for receiving the bit value therefrom, and an output terminal for outputting an inverse of the bit value; and
an AND gate having a first input terminal that is electrically connected to said output terminal of said comparator for receiving the pulse therefrom, a second input terminal that is electrically connected to said output terminal of said inverter for receiving the inverse of the bit value therefrom, and an output terminal that is configured to output the termination indicator in response to the bit value being equal to zero and to receive the pulse from said comparator.

14. The processing device as claimed in claim 11, wherein said PE of the P^{th} one of said processing pairs includes:
an adder having a first input terminal that is configured to receive a value of one, a second input terminal that is electrically connected to said DFF of a (P−1)^{th} one of said processing pairs and that is configured to receive an old diagonal length therefrom, and an output terminal for outputting a new diagonal length as a sum of the old diagonal length and the value of one;
a multiplexer having a first input terminal that is electrically connected to said output terminal of said adder and that is configured to receive therefrom the new diagonal length, a second input terminal that is configured to receive a value of zero, a selector terminal that is electrically connected to said control unit and that is configured to receive the bit value therefrom, and an output terminal that is electrically connected to said SRAM device, and that is configured to output thereto the new diagonal length when the bit value received via the selector terminal is one, and to output thereto the value of zero when the bit value received via the selector terminal is zero;
a comparator having a first input terminal that is electrically connected to said DFF of the (P−1)^{th} one of said processing pairs and that is configured to receive the old diagonal length therefrom, a second input terminal that is configured to receive the preset length threshold, and an output terminal that is configured to output a pulse when the old diagonal length is greater than the preset length threshold;
an inverter having an input terminal that is electrically connected to said control unit and that is configured to receive the bit value therefrom, and an output terminal for outputting an inverse of the bit value; and
an AND gate having a first input terminal that is electrically connected to said output terminal of said comparator and that is configured to receive the pulse therefrom, a second input terminal that is electrically connected to said output terminal of said inverter for receiving the inverse of the bit value therefrom, and an output terminal that is configured to output the termination indicator when the bit value is zero and said comparator outputs the pulse.

15. A system for facilitating variant calling, comprising:
a central processing unit (CPU); and
a distributed processing module electrically connected to said CPU, and including a plurality of first processing devices and a plurality of second processing devices,
wherein said CPU is configured to identify an active region on a reference sequence of deoxyribonucleic acid (DNA) according to a plurality of overlapping reads that are obtained in DNA sequencing, and to offload input data to said first processing devices of said distributed processing module, each of the overlapping reads representing a sequence that overlaps with the active region of the reference sequence of DNA, the input data containing information about the active region of the reference sequence of DNA and the overlapping reads,
wherein said first processing devices of said distributed processing module are configured to generate a de Brujin graph (DBG) based on the overlapping reads and the active region of the reference sequence of DNA, and to perform sequential traversal on the DBG to determine a query sequence of DNA,
wherein said CPU is further configured to obtain information about the query sequence of DNA from said first processing devices of said distributed processing module, and to send the input data and the information about the query sequence of DNA to said second processing devices of said distributed processing module,
wherein said second processing devices of said distributed processing module are configured to perform local alignment to determine a haplotype based on the query sequence of DNA and the reference sequence of DNA, and to determine read-haplotype likelihoods by using a pair hidden Markov model (pair-HMM) forward algorithm (PFA) based on the haplotype and the overlapping reads, and
wherein said CPU is further configured to determine a genotype by using Bayes' Theorem based on the read-haplotype likelihoods thus determined.

16. The system as claimed in claim 15, wherein:
each of said first processing devices of said distributed processing module includes a router for packet switching; and
each of said second processing devices of said distributed processing module includes an L2-cache that is shared among said second processing devices and that is configured to support operations involved in the local alignment and the PFA, and a router for packet switching.

17. The system as claimed in claim 15, wherein:
said CPU is configured to dynamically divide said second processing devices into clusters of various sizes according to sizes respectively of the query sequence of DNA and the reference sequence of DNA, to designate, for each of the clusters, one second processing device in the cluster as a cluster manager, and to distribute tasks to the cluster managers respectively of the clusters;
each of the distributed tasks has a scale that correlates positively with a cluster size of the cluster of the cluster manager that was distributed with the task.

18. The system as claimed in claim 15, wherein:
said distributed processing module includes sixteen of said second processing devices, two of said sixteen second processing devices belonging respectively to two small clusters, six of said sixteen second processing devices belonging to three medium clusters each including two second processing devices, and eight of said sixteen second processing devices belonging to two large clusters each including four second processing devices; and
said CPU is configured to designate one second processing device in each of the small clusters, the medium clusters and the large clusters as a cluster manager, and to distribute a task to the cluster manager of one of the small clusters, the medium clusters and the large clusters according to a scale of the task.
